(19) United States
(12) Patent Application Publication
(10) Pub. No.: US 2005/0170437 A1
(43) Pub. Date: Aug. 4, 2005

(54) Methods and compositions relating to modulating amyloid precursor protein cleavage

(75) Inventors: Luigi Puglielli, Madison, WI (US); Henri J. Huttunen, Boston, MA (US); Suzanne Y. Guenette, Medford, MA (US); Rudolph E. Tanzi, Hull, MA (US); Dora M. Kovacs, Charlestown, MA (US)

Correspondence Address: MaryDilys S. Anderson, Ph.D.;Wolf, Greenfield & Sacks, P.C., 600 Atlantic Avenue, Boston, MA, 02210-2206, US

(73) Assignee: The General Hospital Corporation, Boston, MA (US)

(21) Appl. No.: 10/984,017

(22) Filed: Nov. 8, 2004

Related U.S. Application Data
(60) Provisional application No. 60/518,355, filed on Nov. 7, 2003.

(51) Int. Cl. C12Q 1/68; G01N 33/53; G01N 33/567

(52) U.S. Cl. 435/7.2

(57) ABSTRACT

The invention relates in part assays for identifying and testing compounds that modulate cleavage of amyloid precursor protein (APP). In addition, the invention relates to novel cleavage products of APP. The invention additionally relates to methods and assays for identifying compounds that inhibit acyl-coenzyme A:cholesterol acyltransferase (ACAT) activity. The methods and products of the invention are useful for identifying compounds to prevent and/or treat APP-cleavage associated disorders (e.g. Alzheimer's disease) and are also useful for identifying compounds to prevent and/or treat ACAT-associated disorders.

### RELATED APPLICATIONS

This application claims priority under 35 U.S.C. §119 from U.S. provisional application Ser. No. 60/518,355, filed Nov. 7, 2003.

### FIELD OF THE INVENTION

The invention relates in part to assays for identifying and testing compounds that modulate cleavage of amyloid precursor protein (APP). In addition, the invention relates to novel cleavage products of APP. The invention additionally relates to methods and assays for identifying compounds that inhibit acyl-coenzyme A:cholesterol acyltransferase (ACAT) activity. The invention also relates, in part, to methods and assays for identifying compounds that enhance HtrA2 activity.

### BACKGROUND OF THE INVENTION

Alzheimer's disease (AD) is a neurodegenerative disorder characterized by the abnormal deposition of insoluble protein aggregates in cortical brain regions. Senile plaques constitute the majority of extracellular deposits, and are mainly composed of the amyloid peptide (Aβ) (Glenner, G. G., et al., (1984) Biochem Biophys Res Commun 122: 1131-1135). Aβ is a 39-43 amino acid hydrophobic polypeptide, proteolytically derived from a much larger precursor, the amyloid precursor protein (APP) (Kang, J., et al., (1987) Nature 325: 733-736; Tanzi, R. E., et al., (1987) Science 235: 880-884). For Aβ biogenesis, APP is first cleaved at the N-terminus of Aβ (β-cleavage) by 16-site APP cleaving enzyme 1 (BACE1), producing a 99 amino acid C-terminal fragment (C99) that migrates with the apparent molecular mass of 12-kDa. C99 is subsequently cleaved in the transmembrane domain (γ-cleavage by the presenilin-dependent γ-secretase) releasing Aβ. The two major sites of γ-cleavage are located at positions 40 and 42 of Aβ, generating Aβ40 and Aβ42, respectively. Normally, the 40 amino acid variety represents 90% of secreted Aβ (Selkoe, D. J. (1999) Nature 399: A23-31). APP can also be cleaved between amino acids 16 and 17 of the Aβ sequence (α-cleavage by α-secretases, including cell-surface metalloproteases) generating a C-terminal fragment of ˜9-kDa (C83). α-cleavage of APP does not produce full-length Aβ, but a peptide named p3. Additional minor cleavages by the three major (α-, β-, and γ-secretases within or around the A46 domain of APP have also been reported (Price, D. L., et al., (1998) Annu Rev Genet 32: 461-493; Ling, Y., et al., (2003) Int J Biochem Cell Biol 35: 1505-1535).

In the last few years, cholesterol metabolism has been established as a risk factor for AD by genetic (Sing, C. F., et al., (1985) Am J Hum Genet 37: 268-285; Ehnhohm, C., et al., (1986) J Lipid Res 27: 227-235; Boerwinkle, E., et al., (1987) Am J Med Genet 27: 567-582; Corder, E. H., et al., (1993) Science 261: 921-923; Schmechel, D. E., et al., (1993) Proc Natl Acad Sci USA 90: 9649-9653), epidemiological (Jarvik, G. P., et al., (1995) Neurology 45: 1092-1096; Kuo, Y. M., et al., (1998) Biochem Biophys Res Commun 252: 711-715; Notkola, I. L., et al., (1998) Neuroepidemiology 17: 14-20; Koudinov, A. R., et al., (1998) Clin Chim Acta 270: 75-84; Jick, H., et al., (2000) Lancet 356: 1627-1631; Wolozin, B., et al., (2000) Arch Neurol 57: 1439-1443), and biochemical (Refolo, L. M., et al., (1991) J Neurosci 11: 3888-3897; Lee, S. J., et al., (1998) Nat Med 4: 730-734; Parkin, E. T., et al., (1999) J Neurochem 72: 1534-1543) studies. In addition, both animal (Refolo, L. M., et al., (2000) Neurobiol Dis 7: 321-331; Refolo, L. M., et al., (2001) Neurobiol Dis 8: 890-899; Fassbender, K., et al., (2001) Proc Natl Acad Sci USA 98: 5856-5861) and cellular (Simons, M., et al., (1998) Proc Natl Acad Sci USA 95: 6460-6464; Frears, E. R., et al., (1999) Neuroreport 10: 1699-1705; Fassbender, K., et al., (2001) Proc Natl Acad Sci USA 98: 5856-5861; Puglielli, L., et al., (2001) Nat Cell Biol 3: 905-912) models of AD have shown that cholesterol homeostasis and distribution regulate APP processing and Aβ generation (for a review, see Puglielli, L., et al., (2003) Nat Neurosci 6: 345-351).

Cholesterol is known to modulate the activity of several membrane proteins, including the Sterol Regulatory Element Binding Protein (SREBP) Cleavage-Activating Protein (SCAP) (reviewed in Brown, M. S., et al., (1997) Cell 89: 331-340), the Niemann-Pick type C1 protein (NP-C1) (Carstea, E. D., et al., (1997) Science 277: 228-231), 3-hydroxy-3-methylglutaryl CoA (HMG-CoA)-reductase (Hua, X., et al., (1996) Cell 87: 415-426), and PATCHED, the polytopic receptor for the morphogenic protein Hedgehog (Johnson, R. L., et al., (1996) Science 272: 1668-1671). In addition, cholesterol has been linked to several molecular pathways and interactions. It regulates the proteolytic processing of SREBPs (reviewed in Brown, M. S., et al., (1999) Proc Natl Acad Sci USA 96: 11041-11048) and HMG-CoA reductase (Fitzky, B. U., et al., (2001) J Clin Invest 108: 905-915), modulates the interaction of INSIG-1 with SCAP (Yang, T., et al., (2002) Cell 110: 489-500) and HMG-CoA-reductase (Sever, N., et al., (2003) Mol Cell 11: 25-33), is covalently linked to the Hedgehog protein (Porter, J. A., et al., (1996) Science 274: 255-259), and binds to synaptophysin (Thiele, C., et al., (2000) Nat Cell Biol 2: 42-49) and caveolin (Murata, M., et al., (1995) Proc Natl Acad Sci USA 92: 10339-10343).

Understanding how APP fits into the long list of proteins regulated by cholesterol may shed light on a new aspect of AD pathogenesis. In addition, few treatment options are available for AD or other APP-associated disorders. This lack of clinical options, coupled with the growing number of individuals suffering from diseases such as AD, results in a strong need to identify therapeutic compounds for use in the prevention and treatment of APP-associated disorders such as AD.

### SUMMARY OF THE INVENTION

The invention includes methods for identifying compounds that are useful for treating disorders that are associated with APP processing and the production of β-amyloid (Aβ). Examples of Aβ production-associated disorders include, but are not limited to, Alzheimer's disease and Down's syndrome. We have developed a novel assay that allows identification of compounds that alter amyloid precursor protein (APP) cleavage and we have also devised a novel assay that can be used to identify compounds that modulate the ζ-cleavage activity of HtrA2 and compounds that inhibit acyl-coenzyme A:cholesterol acyltransferase (ACAT) activity. We have unexpectedly found a novel APP cleavage site, which we refer to herein as the ζ-cleavage site of APP. We have also identified that cleavage of APP at the ζ-cleavage site yields novel APP fragments. These APP fragments are useful in the assays and methods of the invention, including assays to identify compounds that alter APP cleavage and assays that identify compounds that modulate (e.g. enhance or inhibit HtrA2 activity) and or compounds that modulate (e.g. inhibit) ACAT activity.

According to one aspect of the invention, methods for identifying compounds that modulate amyloid precursor protein (APP) cleavage are provided. The methods include providing a reaction mixture that comprises APP and/or a fragment thereof that includes a cleavage site, and protein(s) having ζ-cleavage activity, contacting the reaction mixture with a test compound, determining a level of ζ cleavage of APP in the absence and in the presence of the test compound, and comparing the level of ζ cleavage in the absence and in the presence of the test compound, wherein a test compound that increases or decreases the level of ζ cleavage from the level of ζ cleavage in the absence of the test compound is a compound that modulates APP cleavage. In some embodiments, the compound increases the level of ζ cleavage. In certain embodiments, the increase in the level of ζ cleavage is indicative of a reduction in Aβ production. In some embodiments the APP or fragment thereof is a modified APP or fragment thereof. In some embodiments the modified APP or fragment thereof is a polypeptide that is ζ cleavable. In certain embodiments, the APP or fragment thereof is a modified APP or fragment thereof that comprises a ζ-cleavage site and is ζ cleavable. In some embodiments the protein having ζ-cleavage activity is HtrA2. In some embodiments, the level of ζ cleavage is determined using an antibody or antigen-binding fragment thereof that specifically binds to amyloid precursor protein (APP) and/or a fragment of APP. In some embodiments, the antibody is selected from the group consisting of: 22C11, 369, C7, C8, and 6E10. In some embodiments, the fragment of amyloid precursor protein (APP) is a fragment that comprises amino acid 281 and 282 of APP. In certain embodiments, the fragment of amyloid precursor protein (APP) comprises the N terminus of APP or the C terminus of APP. In some embodiments, the N-terminus fragment of APP includes amino acid 281 and the C-terminus fragment of APP includes amino acid 282. In some embodiments, the fragment of amyloid precursor protein (APP) is APPC₄₇₀ or APP_{N1-281}. In certain embodiments, the antibody or antigen-binding fragment thereof is used in an ELISA assay. In some embodiments, the antibody or antigen-binding fragment thereof is used in a Western blot assay. In some embodiments, the reaction mixture is a cell. In some embodiments the compound that modulates ζ-cleavage inhibits or enhances HtrA2 activity. In certain embodiments, the compound that modulates ζ-cleavage inhibits or enhances acyl-coenzyme A:cholesterol acyltransferase (ACAT) activity. In some embodiments, the compound inhibits acyl-coenzyme A:cholesterol acyltransferase (ACAT) activity.

According to another aspect of the invention, an isolated fragment of amyloid precursor protein (APP) that comprises at its C-terminus amino acid 281 of APP is provided.

According to another aspect of the invention, an isolated fragment of amyloid precursor protein (APP) that comprises at its N-terminus amino acid 282 of APP is provided.

According to another aspect of the invention, an isolated fragment of amyloid precursor protein (APP) that comprises amino acid 281 and 282 of APP is provided. In some embodiments, acid 281 and/or amino acid 282 is a mutated amino acid.

According to yet another aspect of the invention, an isolated fragment of a modified APP polypeptide or fragment thereof, which comprises amino acid 281 and 282 of a natural APP polypeptide is provided.

According to another aspect of the invention, an isolated fragment of a modified APP polypeptide that comprises a ζ-cleavage site is provided.

According to yet another aspect of the invention, an isolated fragment of a modified APP polypeptide that is ζ cleavable is provided.

According to another aspect of the invention, methods for identifying polypeptides that are ζ cleavable are provided. The methods include providing a reaction mixture that comprises a candidate polypeptide suspected of being ζ-cleavable, contacting the candidate polypeptide with a protein(s) having ζ-cleavage activity, and determining a level of ζ cleavage of the candidate polypeptide, wherein the presence of ζ cleavage indicates that the candidate polypeptide is ζ cleavable. In some embodiments, the protein having ζ-cleavage activity is HtrA2. In some embodiments, the candidate polypeptide is a modified APP polypeptide or fragment thereof. In certain embodiments, the level of cleavage is determined using an antibody or antigen-binding fragment thereof that specifically binds to amyloid precursor protein (APP) and/or a fragment of APP. In some embodiments, the antibody is selected from the group consisting of: 22C11, 369, C7, C8, and 6E10.

According to another aspect of the invention, methods for preparing an Aβ-associated disease drug are provided. The methods include identifying a compound that increases ζ cleavage activity and formulating the compound for administration to a subject in need of such treatment. In some embodiments, the Aβ-associated disease is selected from the group consisting of: Alzheimer's disease, Down's syndrome, cerebrovascular amyloidosis, Hereditary Amyloidosis with Cerebral Hemorrhage of the Dutch Type, vascular dementia, and inclusion body myositis. In some embodiments, the ζ-cleavage activity is HtrA2 cleavage activity. In some embodiments, the compound that increases ζ cleavage activity is identified by one of the foregoing methods of the invention.

According to another aspect of the invention, kits for identifying compounds that alter amyloid precursor protein (APP) cleavage are provided. The kits include a container containing APP and/or a fragment thereof that includes a ζ-cleavage site, and a container containing a protein that has ζ-cleavage activity. In some embodiments, the protein that has ζ-cleavage activity is HtrA2. In some embodiments, the kit also includes a container containing acyl-coenzyme A:cholesterol acyltransferase (ACAT). In some embodiments, the APP and/or a fragment thereof and the protein that has ζ cleavage activity are contained in a container that is a cell. In some embodiments, the protein that has ζ-cleavage activity is HtrA2. In certain embodiments, the fragment of amyloid precursor protein (APP) is a fragment that comprises amino acid 281 and 282 of APP. In some embodiments, the fragment of amyloid precursor protein (APP) comprises the N terminus of APP or the C terminus of APP. In some embodiments, the N-terminus fragment of APP includes amino acid 281 and the C-terminus fragment of APP includes amino acid 282. In some embodiments, the fragment of amyloid precursor protein (APP) is APPC₄₇₀ or APP_{N1-281}. In certain embodiments, the kit also includes a container containing an antibody or antigen-binding fragment thereof. In some embodiments, the antibody is selected from the group consisting of: 22C11, 369, C7, C8, and 6E10.

These and other aspects of the invention will be described in further detail in connection with the detailed description of the invention.

### BRIEF DESCRIPTION OF THE DRAWINGS

FIG. 1 shows digitized images of Western blots indicating that ACAT inhibition alters the normal proteolytic processing of APP. FIG. 1A shows Western blot analysis of WT and AC29 CHO cells stably transfected with human APP and treated with 250 nM DAPT for the indicated times. Inhibition of secretase activity reveals that generation of C99 and C83 is decreased in AC29 cells, compared to WT CHO cells. FIG. 1B shows Western blot analysis of WT, 25RA and AC29 CHO cell lines stably transfected with APP. Asterisks (*) indicate two APP-CTFs of ˜85- and ˜55-kDa, which are only visible in AC29 cells. The overexposed inset illustrates a marked reduction in C99 and C83 levels in AC29 cells. (Fragments were detected with antibody C7, against the C-terminus of APP.) Values are the mean±SD of at least three separate experiments. #Significant difference vs. control at p<0.05.

FIG. 2 shows histograms and a digitized image Western blot of results indicating that both elevated FC and decreased CE are required for altered proteolytic processing of APP in AC29 cells. AC29 cells stably transfected with APP were treated ad equilibrium with methyl β-cyclodextrin (mβ-CD; 1 mM) and/or mevastatin (5 mM) to reduce FC levels. FIG. 2A shows that only the combination of mβ-CD plus mevastatin reduced FC levels to those found in WT CHO cells. mβ-CD alone was able to reduce but did not normalize FC levels. FIG. 2B shows that normalization of FC increased but did not normalize the secretion of Aβ into the media, reaching ˜50% of total Aβ secreted by WT CHO cells. FIG. 2C shows a Western blot showing that both the ˜55- and ˜85-APP-CTFs disappeared after normalization of FC levels (mg-CD plus mevastatin). This was accompanied by an increase in the steady-state levels of both C99 and C83. (Fragments detected with antibody C7, against the C-terminus of APP. Values are the mean±SD of at least three separate experiments. #Significant difference vs. untreated AC29 cells at p<0.05.)

FIG. 3 shows a schematic diagram of APP₇₅₁ ζ-protease and digitized images of Western blots and histograms indicating that ζ protease cleaves APP at Glu281 and precludes α- and β-cleavages, and Aβ generation. FIG. 3A is a schematic view of APP₇₅₁ illustrating the site of ζ-cleavage. The Aβ region is shown in black and the Kunitz-type proteinase inhibitor (KPI) domain in gray. The dotted lines indicate the single transmembrane (TM) region of APP. FIG. 3B is a Western blot of results obtained when amino acids E281 and S282 were mutagenized to alanine residues to abolish the generation of C470. Western blot analysis of AC29 cells stably transfected with the new construct, APP (ES/AA), shows that mutagenesis of the ζ-cleavage site reactivated C99 and C83 production. Fragments detected with antibody C7, against the C-terminus of APP. FIG. 3C is a histogram that illustrates that AC29 cells stably transfected with APP (ES/AA) fully recovered their ability to generate and secrete Aβ into the media. Aβ levels in the media were detected by sandwich ELISA. (Values are the mean±SD of at least three separate experiments. #Significant difference at p<0.05.)

FIG. 4 shows a schematic diagram of APPs and histograms and a digitized image Western blot indicating that ACAT-mediated regulation of APP processing requires the N-terminus of APP. FIG. 4A are schematic views of APP₇₅₁ (APPwt) and of a N-terminal deletion mutant construct, C470, which lacks amino acids 1-281 and contains a V5/His tag at the C-terminus (APPC470). FIGS. 4B and C show histogram illustrating that AC29 cells stably expressing APPC470 recovered their ability to generate Aβ to normal levels, as shown by a sandwich ELISA. FIG. 4D shows Western blots indicating that APPC470 is processed normally in AC29 cells, in terms of C99 and C83 production. (Fragments detected with antibody: C7, against the C-terminus of APP. Values are the mean±SD of at least three separate experiments. #Significant difference at p<0.05.)

FIG. 5 shows digitized images indicating that ζ-cleavage occurs in an endosomal compartment, followed by proteasomal degradation of C470. FIG. 5A shows a digitized blot indicating results when cell membranes from AC29 cells stably transfected with APP were subjected to an 8-34% Nycomed OptiPrep gradient. Migration of ER, Golgi, and early endosome markers in a typical gradient is shown. FIG. 5B shows a Western blot of APP processing done in pooled fractions to increase the yield. 85-APP-CTF (*) and C470 (>) are indicated; mature (m-APP) and immature (im-APP) APP are also shown. m-APP was only detected in the fractions containing the Golgi marker GM130. Fragments were detected with antibody C7, against the C terminus of APP. FIG. 5C is a Western blot of WT CHO and AC29 cells stably transfected with APP subjected to cell surface biotinylation and Western blot analysis to detect cell surface APP. FIG. 5D is a Western blot that indicates that biotinylation at 15° C. did not reveal C470 generation at the cell surface. FIG. 5E is a Western blot that shows that C470 is produced after internalization of cell surface APP. The cells were replaced at 37° C. following biotinylation. FIG. 5F shows results of AC29 cells stably transfected with APP that were treated with the proteasome inhibitors ALLN (20 mM) or lactacystin (50 mM) and the β-secretase inhibitor Z-VLL-CHO (0.5 mM) for 24 hours prior to SDS-PAGE and Western blot analysis. Proteasome inhibition was able to activate the β cleavage of C470. FIG. 5G shows results of AC29 cells stably transfected with APP that were treated with the lysosomal inhibitors ammonium chloride (NH₄Cl, 10 mM) or chloroquine (100 mM) prior to SDS-PAGE and Western blot analysis. Lysosomal inhibitors did not affect the steady-state levels of C470. (Fragments were detected with antibody C7, against the C-terminus of APP.)

FIG. 6 provides digitized images of Western blots and schematic diagrams of in vitro reconstitution of α-, β-, and ζ-cleavages of APP. FIG. 6A shows Western blot analysis of APP and its CTFs following in vitro reconstitution and incubation for 45 minutes at 35° C. APP was purified from WT CHO cell extracts and reconstituted into intact membranes from untransfected WT CHO or AC29 cells. Membranes were pooled to increase yield. C470, C99, and C83 generation was observed in endosomal, but not ER, membranes. FIG. 6B shows results with reconstituted endosomal vesicles were subjected to chymotrypsin (here shown as scissors) digestion in the presence (FIG. 6C) or absence (FIG. 6B) of Triton X-100 to assess the membrane orientation of APP. APP and C470 were protected from chymotrypsin digestion in the absence of Triton X-100. In contrast, the steady-state levels of both C99 and C83 were found reduced even in the absence of Triton X-100, probably due to three different chymotrypsin digestion sites at the very end of APP's cytosolic tail. (Fragments detected with antibody C7, against the C-terminus of APP.)

FIG. 7 shows digitized images of Western blots and schematic diagrams indicating that one or more membrane proteins regulate the processing of APP by α-, β-, and ζ-secretases. Western blot analysis of APP-CTF generation was done after in vitro reconstitution of APP together with the 350 mM NaCl fraction from the Mono Q-Sepharose column (350 mM NaCl fraction). FIG. 7A shows a Western blot indicating that APP was reconstituted in the absence (lane 1) and presence (lane 2) of additional proteins from the 350 mM NaCl fraction. In lane 3, the 350 mM NaCl fraction was added after reconstitution of APP. Membrane protein(s) from the 350 mM NaCl fraction of the Mono Q-Sepharose column activate ζ-cleavage, while decreasing both ca- and f- cleavages of APP only when reconstituted together with APP. FIG. 7B shows results of purified APPC470 that was reconstituted into native, intact endosomal vesicles in the presence or absence of the 350 mM NaCl fraction. APPC470 served as a substrate for α- and β-secretase cleavage and was unaffected by the 350 mM NaCl fraction. FIG. 7C shows results when APP (ES/AA) was purified and reconstituted into endosomal membrane vesicles in the presence or absence of the 350 mM NaCl fraction. Both C99 and C83 could be detected following in vitro reconstitution of APP (ES/AA) (left lane); the 350 mM NaCl fraction decreased the generation of both C99 and C83 (right lane). (Fragments were detected with antibody: C7, against the C-terminus of APP.)

FIG. 8 shows a schematic model of ACAT-sensitive APP processing.

FIG. 9 is a schematic diagram of the domain structures of HtrA1 and HtrA2 proteases

FIG. 10 is a digitized image of an immunoblot of the ER fraction of AC29 cells that has been stained with rabbit anti-HtrA2 antibodies showing there were increased amounts of mature HtrA2 were found in the ER and Golgi fractions of AC29 as compared to the corresponding fractions from wild-type CHO cells. The first lane contains an aliquot from a partially purified APP₇₅₁ sample that was resolved on the same gel. The staining show that HtrA2 co-purifies with APP₇₅₁ in ion exchange chromatography.

FIG. 11 is a digitized image of an immunoblot of an AC29 cell preparation that demonstrates the localization of HtrA2 in AC29 cells.

### DETAILED DESCRIPTION OF THE INVENTION

We have identified a novel cleavage site of amyloid precursor protein (APP), which we have named “ζ cleavage” (zeta cleavage) and we have discovered that an increase in ζ cleavage of APP results in a decrease in the production of A8. We have also identified that the protease HtrA2 cleaves APP at the ζ cleavage site. The invention includes in some aspects an assay to identify candidate agents that modulate ζ cleavage of amyloid precursor protein. Thus, the invention relates to assays to identify candidate agents that modulate Aβ production. We have also discovered that a decrease in ACAT activity increases ζ cleavage of APP and results in a concomitant decrease in the production of Aβ. Thus, some aspects of the invention include assays to identify candidate agents that modulate ACAT activity. As used herein, the term “APP” includes APP splice forms. Examples of splice forms of APP include, but are not limited to, APP695 and APP751. The 770 amino acid sequence of human APP peptide is provided as Genbank Accession No. P05067, and the entry includes sequence information on a number of APP splice forms. The Genbank Accession numbers for the APP751 splice form of human APP include X06989 and Y00297.

As used herein, the term “ζ cleavage” means ζ cleavage of a protein by protease at a ζ-cleavage site. In some embodiments of the invention, the protein is APP. As used herein the term “ζ cleavable” means the ability to undergo ζ cleavage. For example, a polypeptide that is cleavable is a polypeptide that under conditions suitable for ζ cleavage is ζ cleaved. ζ cleavage of APP means cleavage of APP at amino acids equivalent to E281 and S282 of the 751 amino acid splice form of APP. Thus, an APP peptide that is useful in the invention is an APP peptide or variant thereof that includes the amino acid residues that correspond to E281 and S282 of APP751, (e.g. has a ζ-cleavage site). ζ cleavage of the 751 amino acid splice form of APP generates a 55 kDa C-terminal fragment of APP that contains the last 470 amino acids of the 751 splice-form of APP, which is referred to herein as “APP_{C470}”, “C470”, and the like. As used herein the term “APP_{N1-281}” means an N-terminal fragment of APP that includes amino acids 1-281 and is generated by ζ cleavage of APP. A second C-terminal fragment generated by ζ cleavage of APP is an 85 kDa C-terminal fragment. As used herein, the terms “ζ-secretase activity” and “ζ-protease activity” mean the function of ζ cleavage of APP. As shown in the Examples section, a proteinaceous factor is involved in ζ cleavage of APP. As used herein, the factor that cleaves APP at the ζ-cleavage site is also referred to as ζ-secretase and/or ζ protease. An example of a protein with ζ cleavage activity is HtrA2.

The invention includes assays to identify compounds that prevent and/or reduce the production and/or accumulation of Aβ in Alzheimer's disease and other disorders. As used herein, the terms “Aβ-associated disorder” and “Aβ production-associated disorders” and “ζ-cleavage associated disorders” include, but are not limited to: Alzheimer's disease, Down's syndrome, cerebrovascular amyloidosis, Hereditary Amyloidosis with Cerebral Hemorrhage of the Dutch Type, vascular dementia, and inclusion body myositis.

The invention includes methods for screening for compounds that modulate amyloid precursor protein (APP) cleavage and/or agents that modulate acyl-coenzyme A:cholesterol acyltransferase (ACAT) activity. Such methods include cell based (in vitro and in vivo) and non-cell based assays of various kinds. For example, non-cell based assays can involve combining cell extracts from cells that have APP or a fragment thereof that includes a ζ cleavage site and is ζ cleavable, with a protein or proteins having ζ-secretase activity (e.g. HtrA2) and contacting the mixture with compounds that are candidate modulators of ζ cleavage. Cell-based assays can include contacting a cell that has APP or a fragment thereof that includes a ζ cleavage site and a protein or proteins having ζ-secretase activity (e.g. HtrA2) with compounds that are candidate modulators of ζ cleavage. As used herein, a protein with secretase activity is a protein with a protease activity, e.g. a protein that functions to cleave APP at a specific cleavage site. For example, a ζ protease or secretase cleaves APP at the ζ-cleavage site and a γ protease or secretase cleaves APP at the γ cleavage site. HtrA2 is an example of a ζ protease that cleaves APP at the ζ-cleavage site.

As described above, the invention relates in some aspects to the identification and testing of candidate ζ cleavage-modulating compounds. In some embodiments, ζ cleavage modulating compounds are ACAT activity-modulating compounds. Candidate cleavage-modulating compounds can be screened for modulating (enhancing or inhibiting) ζ cleavage of APP, ACAT activity, and/or Aβ production using the assays described herein (e.g., in the Example section). Using such assays, the ζ cleavage-modulating compounds that have ζ-cleavage enhancing or inhibiting activity can be identified. It is understood that any mechanism of action described herein for the ζ cleavage-modulating compounds is not intended to be limiting, and the scope of the invention is not bound by any such mechanistic descriptions provided herein.

The invention further provides efficient methods of identifying pharmacological agents or lead compounds for agents and compounds that modulate ζ cleavage of APP, HtrA2 activity, ACAT activity, and/or Aβ production. Generally, the screening methods involve assaying for compounds which modulate (enhance or inhibit) the level of ζ cleavage of APP, HtrA2 activity, ACAT activity, and/or Aβ production. As will be understood by one of ordinary skill in the art, the screening methods may measure the level of ζ cleavage of APP directly, e.g., using the screening methods described herein.

A wide variety of assays for pharmacological agents can be used in accordance with this aspect of the invention, including, ζ cleavage assays, ACAT activity assays, HtrA2 activity assays, Aβ production assays, cell viability assays, cell-based assays, non-cell based assays, etc. As used herein, the term “pharmacological agent” means ζ cleavage-modulating, HtrA2 activity modulating, and/or ACAT activity modulating compounds. An example of such an assay that is useful to test candidate ζ cleavage-modulating and/or ACAT activity modulating compounds is provided in the Examples section. In such assays, the assay mixture comprises a candidate pharmacological agent. Typically, a plurality of assay mixtures is run in parallel with different agent concentrations to obtain a different response to the various concentrations. Typically, one of these concentrations serves as a negative control, i.e., at zero concentration of agent or at a concentration of agent below the limits of assay detection.

Some aspects of the invention include non-cell based assays in which isolated APP polypeptide or a fragment thereof that includes a ζ cleavage site, or cell extracts from cells that have APP polypeptide or a fragment thereof that includes a ζ cleavage site are combined with a polypeptide or polypeptides having ζ-secretase activity, (e.g. HtrA2), and the mixture is contacted with compounds that are candidate modulators of ζ cleavage. In some embodiments, ACAT is added to a reaction mixture in an assay of the invention. In some embodiments, a reaction mixture containing APP polypeptide or a fragment thereof that includes a ζ cleavage site, and a polypeptide or polypeptides having ζ-secretase activity is contacted with ACAT and compounds that are candidate modulators of ζ cleavage. In the non-cell-based assays of the invention, the assay components, e.g. the polypeptide or fragment thereof with ζ-secretase activity, and/or the APP polypeptide with the ζ cleavage site can be from one or more cell extracts or can be in isolated form.

Some aspects of the invention include cell-based assays in which cells that have APP polypeptide or a fragment thereof that includes a ζ-cleavage site and a polypeptide or polypeptides having ζ-secretase activity may be contacted with compounds that are candidate modulators of ζ cleavage. In some embodiments, the polypeptide having ζ-secretase activity is HtrA2. For cell-based and non-cell-based assays of the invention, compounds that modulate (either inhibit or enhance) ζ-secretase activity may be identified by determining the level of ζ cleavage of APP, relative to a control cell or cell extract (e.g., a control that is not contacted with a candidate compound, or a control assay not contacted with ACAT and a candidate compound).

The invention includes the use of the aforementioned polypeptides in assays to identify compounds that modulate levels of ζ cleavage of APP, modulate levels of HtrA2 activity, or that modulate levels of ACAT activity. The assays of the invention include, but are not limited to assays of the type described in the Examples, and include in vitro protein-protein binding assays, Western blot assays, electrophoretic mobility shift assays, immunoassays, cell-based assays such as two- or three-hybrid screens, expression assays, etc. The assay mixture comprises a candidate pharmacological agent, e.g. a candidate ζ cleavage modulator and/or a candidate ACAT activity modulator. The various assays used to determine the levels of ζ cleavage of APP and/or ACAT activity levels include the use of materials that specifically bind to APP, materials that specifically bind to fragments of APP, gel electrophoresis; and the like. Immunoassays may be used according to the invention including sandwich-type assays, competitive binding assays, one-step direct tests and two-step tests such as routinely practiced by those of ordinary skill in the art. Typically, a plurality of assay mixtures is run in parallel with different candidate modulator concentrations to obtain a different response to the various concentrations. Typically, one of these concentrations serves as a negative control, i.e., at zero concentration of agent or at a concentration of agent below the limits of assay detection. It is contemplated that cell-based assays as described herein can be performed using cell samples and/or cultured cells. Cells of the invention include cells treated using methods described herein to modulate (e.g. inhibit or enhance) the level of ACAT activity, and/or the level of HtrA2 activity, and/or ζ cleavage of APP.

The candidate ζ cleavage-modulating molecules used in the assays of the invention can be natural or synthetic compounds, such as those in small molecule libraries of compounds (including compounds derived by combinatorial chemistry). Natural product libraries also can be screened using such methods, as can selected libraries of compounds known to exert pharmacological effects, such as libraries of FDA-approved drugs. Compounds identified by the assays can be used in therapeutic methods of the invention described below.

Candidate ζ cleavage-modulating molecules of the invention encompass numerous chemical classes, although typically they are organic compounds. In some embodiments, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate ζ cleavage-modulating molecules of the invention are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents can be tested and further may be subjected to directed or random chemical modifications such as acylation, alkylation, esterification, amidification, etc. to produce structural analogs of the agents.

The ζ cleavage-modulating molecules of the invention may include small molecules, polypeptides, (for example, competitive ligands and antibodies, or antigen-binding fragments thereof), and nucleic acids. For example, compositions of the invention may include nucleic acids that encode a molecule that increase the transcription of HtrA2 and/or reduces transcription of ACAT biosynthetic enzymes, including nucleic acids that bind to other nucleic acids, [e.g. for antisense, RNAi, or small interfering RNA (siRNA) methods], or may be polypeptides that increase the levels or activity of HtrA2 and/or reduce the levels or activity of ACAT. Such polypeptides include, but are not limited to antibodies or antigen-binding fragments thereof.

A variety of other reagents also can be included in the assay mixtures of the invention. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The assays of the invention may be used to identify candidate agents that modulate 1) production of ζ cleavage products of APP, 2) the ζ cleavage of APP, 3) the activity of HtrA2, and/or 4) the activity of ACAT. As used herein, the term “modulate” means to change, which in some embodiments means to “enhance” or “increase” and in other embodiments, means to “inhibit” or “reduce”. For example, in some embodiments, ζ cleavage of APP enhanced or increased. In some embodiments, cleavage activity of HtrA2 is increased or enhanced. In other embodiments, ACAT activity is inhibited or reduced. It will be understood that reduction may mean reduction to zero or may mean reduction to a level below a normal level, a previous level, or a control level.

In general, the mixture of the foregoing assay materials is incubated under conditions whereby, but for the presence of the candidate pharmacological agent, a control level of ζ cleavage of APP will occur (e.g., little or no ζ cleavage of APP), and/or a control level of HtrA2 activity will occur, and/or a control level of ACAT activity will occur. It will be understood that a candidate pharmacological agent that is identified as a modulating agent may be identified as increasing ζ cleavage of APP. An increase in ζ cleavage of APP may mean an increase from zero ζ cleavage of APP or may be an increase from a control level of ζ cleavage to a higher level of ζ cleavage. It will be understood that a candidate pharmacological agent that is identified as a modulating agent may be identified as decreasing or eliminating ACAT activity. A reduction in ACAT activity need not be the absence of ACAT activity, but may be a lower level of ACAT activity than in a control.

It will be understood that a candidate pharmacological agent that is identified as a modulating agent may be identified as increasing HtrA2 activity. An increase in HtrA2 activity may mean an increase from zero activity or may be an increase from a control level of ζ cleavage to a higher level of ζ cleavage.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the level of ζ cleavage of APP, the activity of ACAT, that activity of HtrA2, and/or the production of Aβ may be detected by any convenient method available to the user. One method of detection that is useful in the methods of the invention is the use of polypeptides, (e.g. antibodies), that specifically bind to APP, regions of APP, specific fragments of APP, and/or Aβ or specific regions of Aβ. Detection may be effected in any convenient way for the assays of the invention. For example, an antibody may be coupled to a detectable label. For cell-based assays, one of the assay components may comprise, or be coupled to, a detectable label. A wide variety of detectable labels can be used, such as those that provide direct detection (e.g., radioactivity, luminescence, optical or electron density, etc.) or indirect detection (e.g., epitope tag such as the FLAG epitope, enzyme tag such as horse-radish peroxidase, etc.).

A variety of methods may be used to detect the label, depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

The assays described herein may be carried out on samples obtained from subjects. As used herein, a subject is a human, non-human primate, cow, horse, pig, sheep, goat, dog, cat, or rodent. In all embodiments, human subjects are preferred. The samples used herein may be any cell, body tissue, or body fluid sample obtained from a subject. In some embodiments, the cell or tissue sample includes neuronal cells and/or is a neuronal cell or tissue sample.

The cells utilized in the assays of the invention, can be located in vitro (e.g., a biopsy such as a tissue biopsy or tissue extract) and can be part of a cell-containing biological sample. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, a cerebrospinal fluid sample, a tissue biopsy, including punch biopsy, a tumor biopsy, a bodily fluid, a tissue, or a tissue extract or other methods. Samples also can be cultured cells, tissues, or organs. In some embodiments of the invention, the cells are in vivo.

The assays described herein (see, e.g., the Examples section) include measuring the level of ζ cleavage of APP, HtrA2 activity, and/or ACAT activity. Levels of ζ cleavage of APP, HtrA2 activity, and/or ACAT activity can be measured in a number of ways when carrying out the various methods of the invention. In one type of measurement, the level of ζ cleavage of APP is a measurement of the level of the products of ζ cleavage of APP, e.g., the level of APPC₄₇₀ or APP_{N1-281}. This could be expressed, for example, in terms of molecules per cubic millimeter of tissue. Another measurement of the level of ζ cleavage of APP may be the measurement of the level of products of non-ζ cleavage of APP, e.g. a and/or 0 cleavage products, and/or Aβ. In addition, another measure of the level of ζ cleavage may be in the reduction of the level of APP. These measurements of cleavage product levels may be expressed in an absolute amount or may be expressed in terms of a relative level of one cleavage product versus another cleavage product. For example, a reduction in α and/or β cleavage and an increase in ζ cleavage may be compared relative to each other as a measure of the level of modulation of ζ cleavage activity in response to a candidate agent.

Importantly, levels of ζ cleavage of APP, activity of HtrA2, and/or ACAT activity are advantageously compared to controls according to the invention. The control may be a predetermined value, which can take a variety of forms. It can be a single value, such as a median or mean. It can be established based upon comparative groups (e.g. comparative cell types), such as in cells having normal amounts of ζ cleavage of APP (which may be no ζ cleavage of APP), normal levels of HtrA2 activity, and/or normal levels of ACAT activity. The determination of the levels of ζ cleavage of APP and the determination of the level of HtrA2 and/or ACAT activity correlate with the level of cleavage products (e.g. level of APP_{C470} or APP_{N1-281}) in a particular experimental system. Another example of comparative cell types would be cells from subjects known to have a particular disease (e.g., Alzheimer's disease), condition or symptoms, and cells from groups without the disease, condition or symptoms. Another comparative cell type would be cells from subjects with a family history of a disease or condition and a group without such a family history.

The predetermined value of course, will depend upon the particular population of cells selected. For example, an apparently healthy cell population will have a different ‘normal’ range of ζ cleavage, HtrA2 activity, and/or ACAT activity than will a population that is known to have a condition related to APP cleavage or processing or a condition related to abnormal levels (e.g. different from control levels) of HtrA2 activity and/or ACAT activity. Accordingly, the predetermined value selected may take into account the category in which a cell type falls. Appropriate ranges and categories can be selected with no more than routine experimentation by those of ordinary skill in the art. By abnormal levels it is meant abnormal (high or low) relative to a selected control. Typically the control will be based on apparently healthy normal cell types.

It will also be understood that the controls according to the invention may be, in addition to predetermined values, samples of materials tested in parallel with the experimental materials. Examples include samples from control cells or control samples (e.g., generated through manufacture) to be tested in parallel with the experimental samples.

As mentioned above, it is possible to determine the efficacy of a candidate compound to modulate APP processing (e.g. APP cleavage) and/or to modulate HtrA2 activity and/or ACAT activity by monitoring changes in the absolute or relative amounts of APP and/or specific fragments of APP (e.g. APPC₄₇₀, APP_{N1-281}) in the absence and/or presence of a candidate compound. For example, an increase in the amount of fragments such as level of APP_{C470} or APP_{N1-281} indicate that a candidate compound increased ζ cleavage of APP, thus reducing α, β, and/or γ cleavage of APP. The ratio of ζ cleavage to α, β, and/or γ cleavage provides an indication of the efficacy of a candidate compound's enhancement of ζ cleavage of APP. It will be understood that a higher level of ζ cleavage of APP in a cell correlates to a lower level of Aβ generation by the cell. Similarly, a lower level of ζ cleavage of APP in a cell correlates to a higher level of Aβ production by the cell. Accordingly, one can monitor levels of ζ cleavage of APP and/or levels of Aβ to determine the efficacy of a candidate compound tested in an assay of the invention. Thus, using the assays of the invention, one can identify compounds for use in the prevention and/or treatment of conditions associated with abnormal Aβ production.

Changes in relative or absolute levels of ζ cleavage of APP, HtrA2 activity, and/or ACAT activity of greater than 0.1% when contacted with a candidate compound in an assay of the invention may indicate a compound that is effective for the prevention and/or treatment of a condition associated with abnormal Aβ production. Preferably, the change in levels of ζ cleavage of APP, HtrA2 activity, and/or ACAT activity, which indicates a compound is effective, is greater than 0.2%, greater than 0.5%, greater than 1.0%, 2.0%, 3.0%, 4.0%, 5.0%, 7.0%, 10%, 15%, 20%, 25%, 30%, 40%, 50%, or more. As described above, an increase in ζ cleavage of APP, increase in HtrA2 activity, and/or a decrease in ACAT activity in an assay of the invention, both indicate a compound that may be used to prevent and/or treat a condition associated with abnormal Aβ production. It will be understood that in some cases, when the initial level of ζ cleavage of APP is zero, the change in the level that indicates that a compound is effective, will be any level greater than zero, with higher levels indicating higher effectiveness.

The invention includes the use of agents (e.g., antibodies or antigen-binding fragments thereof) to determine the level of ζ cleavage of APP, HtrA2 activity, and/or ACAT activity in the assays of the invention. As used herein, the term “antibodies” includes antibodies or antigen-binding fragments thereof. Antibodies of the invention can be identified and prepared that bind specifically to APP and/or APP fragments generated by ζ cleavage of APP. Fragments generated by ζ cleavage of APP may include: APP_{C470}, APP_{N1-281}, a fragment of APP that comprises at its C-terminus amino acid 281 of APP, or a fragment of APP that comprises at its N-terminus amino acid 282 of APP. The methods of the invention also may include use of an antibody that specifically binds to a APP or a fragment of APP that comprises amino acid 281 and 282 of APP. In some embodiments, such as when a ζ-cleavage substrate of a natural amino acid seqeunce is used, a fragment may be a fragment that has a mutated amino acid sequence. For example, in some embodiments of the invention, amino acid 281 and/or amino acid 282 of an APP fragment is a mutated amino acid and is not the wild-type E281 and/or S282. As used herein the term “mutated amino acid” means an amino acid that differs from the amino acid in the same sequence position in the wild-type peptide. Therefore, in some embodiments, an antibody of the invention specifically binds to a mutated APP or fragment thereof, e.g. specifically binds to APP or a fragment thereof with a mutated amino acid 281 and/or 282.

As used herein, “binding specifically to” means capable of distinguishing the identified material from other materials sufficient for the purpose to which the invention relates. Thus, “binding specifically to” a fragment of APP means the ability to bind to and distinguish these molecules from other proteins.

Examples of antibodies that are useful in the methods of the invention include, but are not limited to: 22C11 (monoclonal, against N-terminus of APP); 369, C7, and C8 (monoclonal, against C-terminus of APP); 4G8 (monoclonal, against residues 18-25 of Aβ); And 6E10 (monoclonal, against residues 1-17 of Aβ). Antibodies that are useful in methods of the invention include antibodies that specifically bind to APPC₄₇₀, antibodies that specifically bind to APP_{C1-281}. The antibodies of the invention can be used to identify the presence of fragments of APP generated by ζ cleavage and therefore are useful in the assays of the invention. The methods of the invention also include in some embodiments, antibodies that bind specifically to fragments of APP that are generated through α, β and/or γ cleavage of APP. For example, antibodies that specifically bind to Aβ (e.g. 4G8 and 6E10). Such antibodies are useful in the methods of the invention to determine the amount of APP processing that is α, β, and/or -γ cleavage, which can indicate the level of Aβ (as an indicator of ζ-cleavage levels) and/or can be used for comparison to the amount of APP cleavage that is cleavage.

One of ordinary skill will recognize that antibodies that specifically bind to APP, APP regions, or specific fragments of APP can be used to determine the presence of absence of various regions and/or fragments of APP in the assays of the invention. Methods of using various antibodies that specifically bind to regions of APP, fragments of APP, Aβ, or regions of Aβ etc. to determine the cleavage of APP in assays of the invention are described in the Examples section. The antibodies and antigen-binding fragments thereof of the invention can be used for the assay of ζ cleavage modulators, HtrA2 activity modulators, and/or ACAT activity modulators using known methods including, but not limited to enzyme linked immunosorbent (ELISA) assays, immunoprecipitations, and Western blots.

The antibodies of the present invention may be prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies or antigen-binding fragments thereof may be used for example to identify the presence of specific fragments of APP, e.g., the presence of Aβ or APP_{C470} as an indication of the efficacy of a candidate compound for enhancing ζ cleavage of APP. The antibodies of the invention include monoclonal and polyclonal antibodies.

Antibodies also may be coupled to specific labeling agents, for example, for imaging of cells and tissues with ζ cleavage of APP according to standard coupling procedures. Labeling agents include, but are not limited to, fluorophores, chromophores, enzymatic labels, radioactive labels, etc. Other labeling agents useful in the invention will be apparent to one of ordinary skill in the art.

Significantly, as is well known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd Fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of murine RSV antibodies in which at least a portion of the murine FR regions have been replaced by FR regions of human origin. Such antibodies, including fragments of intact antibodies with antigen-binding ability, are often referred to as “chimeric” antibodies. Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)2, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to an APP molecule, specific regions of an APP molecule, and/or to specific fragments of APP. The polypeptides may be derived also from sources other than antibody technology. For example, such polypeptide-binding agents can be provided by degenerate peptide libraries, which can be readily prepared in solution, in immobilized form or as phage display libraries. Combinatorial libraries also can be synthesized of peptides containing one or more amino acids. Libraries further can be synthesized of peptoids and non-peptide synthetic moieties.

The methods of the invention can be used to screen or identify various compounds that are useful to decrease Aβ production. Aβ production may be decreased, e.g., for treatment of Alzheimer's disease, using methods to decrease the level of ACAT activity and/or methods to increase the level of ζ cleavage of APP, and/or methods to increase the activity of HtrA2. For example, the assays of the invention can be used to assess the efficacy of increasing HtrA2 activity that include 1) contacting a cell with molecules that increase HtrA2 activity or production of HtrA2. For example, HtrA2 and/or nucleic acid molecules that encode HtrA2 may be administered to increase the presence of HtrA2 in a cell, tissue, or subject. The methods of increasing activity of the proteins in the production of HtrA2 may also include administering polypeptides or nucleic acids that encode polypeptides that are variants of the HtrA2 molecules that are functional variants. Such variants may compete with the supplement functional endogenous versions in a cell, tissue, or subject, and thereby increase the HtrA2 activity and reduce the level of Aβ in cells.

In addition, the assays of the invention can be used to assess the efficacy of reducing ACAT activity that include 1) contacting a cell with molecules that are antisense of the nucleic acids that encode ACAT, 2) RNAi and/or siRNA inhibition methods of ACAT, and/or 3) administration of antibodies that block the functional activity of the proteins in the production of ACAT. The methods of reducing activity of the proteins in the production of ACAT may also include administering polypeptides or nucleic acids that encode polypeptides that are variants of the ACAT molecules that are not functional or are not fully functional. Such variants may compete with the functional endogenous versions in a cell, tissue, or subject, and thereby reduce the ACAT activity and the production of Aβ in cells. Compounds that modulate ζ cleavage of APP, HtrA2 activity-modulating compounds, and/or ACAT activity-modulating compounds of the invention, which include for example, antisense oligonucleotides, RNAi and/or siRNA oligonucleotides, antibodies, nucleic acids, an/or polypeptides may be administered as part of a pharmaceutical composition.

One set of embodiments of the aforementioned compositions and methods include the use of antisense molecules or nucleic acid molecules that reduce expression of genes via RNA interference (RNAi or siRNA). One example of the use of antisense, RNAi or siRNA in the methods of the invention is their use to decrease the level of expression of one or more ACAT biosynthetic pathway enzymes. The antisense oligonucleotides, RNAi, or siRNA nucleic acid molecules used for this purpose may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods, which may be carried out manually or by an automated synthesizer. They also may be produced recombinantly by vectors.

In some embodiments of the invention, the antisense or siRNA oligonucleotides also may include “modified” oligonucleotides. That is, the oligonucleotides may be modified in a number of ways, which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars that are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose.

Some aspects of the invention include kits for assaying for compounds that modulate cleavage of APP. Some aspects of the invention include kits for assaying for compounds that modulate HtrA2 activity. Some aspects of the invention include kits for assaying for compounds that modulate ACAT activity. Kits of the invention may include APP and a polypeptide with ζ cleavage activity. In some embodiments, the polypeptide with ζ cleavage activity is HtrA2. Kits of the invention may also include control solutions or molecules for use in the assays of the invention.

Kits of the invention may also include molecules that bind to APP or fragments thereof. The binding molecules may be antibodies or antigenic-fragments thereof and may be detectably labeled. As described herein, the binding molecules may be monoclonal or polyclonal antibodies that specifically bind to APP or fragment thereof. The kit may also include materials for processing using procedures well known to those of skill in the art, to assess whether specific binding occurred between the APP and/or fragments thereof and agents (e.g. antibodies) in the assay mixture. For example, procedures may include, but are not limited to, contact with a secondary antibody, or other method that indicates the presence of specific binding. The foregoing kits may also include instructions or other printed material on how to use the various components of the kits for identifying compounds that modulate ζ cleavage of APP, HtrA2 activity, ACAT activity, and/or Aβ production.

The polypeptides of the invention also include polypeptide fragments. APP fragments may include APP polypeptide fragments, which are APP polypeptides that are not full-length APP polypeptides. For example, a fragment of APP may have an amino acid sequence that is the sequence of APP reduced in length at one and/or both ends by up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, or more amino acids (including every integer therebetween), as long as the APP fragment retains a functional capability of APP. The variants may be identified as retaining a functional capability [e.g. the ability to be ζ cleaved (ζ cleavable), antibody binding capabilities useful in the assays] of the APP or APP fragment polypeptides. An example of a functional fragment of APP that may be used in the methods of the invention is a fragment of APP that includes at least amino acid E281 and S282 of APP. Another example of a functional fragment of APP that may be used in the methods of the invention is a fragment of APP that is ζ cleavable.

It will be understood that the polypeptides used in the assays of the invention also include modified polypeptides, including but not limited to modified APP polypeptides or APP fragments, modified polypeptides with ζ activity, modified HtrA2 peptides, modified ACAT polypeptides, etc. Thus, the invention includes use of modified polypeptides in the assays of the invention. The modified polypeptides include polypeptides having single amino acid changes from the normal (wild-type) amino acid sequence of a polypeptide of the invention can be prepared. Likewise, modified polypeptides of the invention include polypeptides having 2, 3, 4, 5, 6, 7, 8, 9, 10, 11, 12, 13, 14, 15, 16, 17, 18, 19, 20, or more amino acid changes. Numerous modified polypeptide molecules like these will be readily envisioned by one of skill in the art. Some of the modified polypeptides of the invention are modified APP polypeptides that are recognized by agents (e.g. antibodies) that specifically bind to APP and/or specific APP fragments described above herein. Other modified polypeptides of the invention are modified proteins that are ζ-cleavable. As used herein, the term “ζ-cleavable” means that the polypeptide can be ζ cleaved. Still other modified polypeptides of the invention are those associated with ACAT activity. Still other modified polypeptides of the invention are those associated with HtrA2 activity. The descriptions of modifications set forth below exemplify modification of APP polypeptides or fragments thereof, but it will be understood by one of ordinary skill in the art that the exemplified modifications may also occur in other polypeptides utilized in methods, kits and compositions of the invention.

In addition to wild-type (e.g., natural) polypeptides and fragments thereof that are useful in the invention, variant or modified polypeptides and/or fragments that are useful in the methods and kits of the invention can be identified by one of ordinary skill in the art. In some embodiments, the modified polypeptide is a modified APP polypeptide or fragment thereof As used herein the term “modified APP polypeptide or fragment thereof” means an APP polypeptide that has one or more modifications in amino acid sequence from the amino acid sequence of a natural (wild-type) APP or fragment thereof. One of ordinary skill will understand that a modified APP polypeptide or fragment thereof may have an addition, deletion, substituted, or altered amino acid, or any combination of these types of amino acid changes. For example, an APP polypeptide or fragment thereof that is useful in the claimed methods of the invention may be an APP polypeptide or fragment that comprises a ζ-cleavage site, is ζ cleavable, but has a region of its non-ζ-cleavage site sequence added to, deleted, or substituted with a sequence that is different than the natural (e.g. wild-type) sequence.

In addition, a modified APP polypeptide or fragment thereof of the invention may comprise a ζ-cleavage site, be ζ cleavable, and also include one or more amino acids that are additional within (or at either or both ends of) the natural sequence of an APP polypeptide or fragment thereof. For example, an APP polypeptide of the invention may be a modified polypeptide that includes all or part of the sequence of APP751 with one or more additional amino acids included in the sequence between the natural amino acid 1 and amino acid 751. Such a modified AP751 may also include one or more additional amino acids at one or both ends of the APP751 polypeptide sequence.

In some embodiments of the invention, a polypeptide of the invention may be a fusion protein. For example, an APP polypeptide of the invention may be an APP fusion protein that includes a natural or modified APP polypeptide sequence or fragment thereof and may also include one or more additional amino acids that do not occur adjacent to or in conjunction with a natural (wild-type) APP sequence. One of ordinary skill in the art will understand how to prepare and test fusion proteins for use in the methods of the invention.

The skilled artisan will realize that some modified polypeptides of the invention may have conservative amino acid substitutions, and that conservative amino acid substitutions may be made in the polypeptides of the invention and still allow their use in the methods of the invention. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the polypeptides of the invention include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

It will be understood that one can make a conservative substitution to the amino acid sequence of a polypeptide of the invention and still have it be functional in the assays of the invention. For example, a conservative substitution can be made to the amino acid sequence of an APP polypeptide or fragment thereof and still have the polypeptide retain its functional activity (e.g., ability to be ζ cleaved and/or specific antibody-binding characteristics), and thus its usefulness in the assay methods of the invention. Conservative amino-acid substitutions in the amino acid sequence of a component of an assay of the invention, including, but not limited to APP polypeptide and/or APP fragments, a polypeptide with ζ activity, an ACAT polypeptide, an HtrA2 polypeptide to produce functionally equivalent variants of the molecules of the invention may be made by alteration of a nucleic acid encoding a polypeptide of the invention. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492: 1985), or by chemical synthesis of a gene encoding an APP polypeptide. Where amino acid substitutions are made to a small unique fragment of an APP polypeptide, the substitutions can be made by directly synthesizing the peptide. The activity of functionally equivalent fragments of APP polypeptides can be tested by cloning the gene encoding the altered APP polypeptide into a bacterial or mammalian expression vector, introducing the vector into an appropriate host cell, expressing the altered polypeptide, and testing for a functional capability of the APP polypeptide in the assays of the invention. Peptides that are chemically synthesized can be tested directly for function, e.g., ability to be cleaved and/or ability to bind to antibodies of the invention.

The invention includes in some aspects, methods of testing candidate polypeptides for their ability to ζ cleaved. For example, one such method of the invention for determining whether a candidate polypeptide is ζ cleavable includes providing a reaction mixture that includes a candidate polypeptide suspected of being ζ-cleavable, contacting the polypeptide with a protein(s) having ζ-cleavage activity, and determining a level of ζ cleavage of the candidate polypeptide. The level of ζ cleavage of a candidate polypeptide can be determined by assessing the presence of full-length candidate polypeptide and/or cleavage products in the reaction mixture, and can also be assessed by detection the absence of specific APP cleavage products (ζ-, α- and/or β-cleavage products) in the reaction mixture.

The polypeptides useful in accordance with the invention, and fragments thereof, can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. As used herein, the term “polypeptide” means polypeptide and protein. Short polypeptides also can be synthesized chemically using well-established methods of peptide synthesis.

Thus, as used herein with respect to proteins, “isolated” means separated from its native environment and present in sufficient quantity to permit its identification or use. Isolated, when referring to a protein or polypeptide, means, for example: (i) selectively produced by expression of a recombinant nucleic acid or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure proteins may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, e.g. isolated from other proteins.

The invention includes in some aspects the use of compounds that modulate ζ cleavage of APP, which are identified using the assays of the invention, in therapeutic methods for the prevention and treatment of conditions associated with abnormal All production. These methods of the invention include administration of ζ cleavage-modulating compounds, to increase the level of ζ cleavage of APP in cells or tissues, and thereby to decrease Aβ production. In some embodiments, the ζ-cleavage modulating compounds will be HtrA2 activity-modulating compounds. HtrA2 activity-modulating compounds may increase the level of HtrA2 activity in cells or tissues, and thereby increase ζ cleavage of APP and decrease Aβ production.

In some embodiments, the ζ-cleavage modulating compounds will be ACAT activity-modulating compounds. ACAT activity-modulating compounds may decrease the level of ACAT activity in cells or tissues, and thereby increase ζ cleavage of APP and decrease Aβ production.

In general, the treatment methods of the invention involve administering an agent to modulate the level and/or activity of ζ cleavage of APP, HtrA2 activity, and/or ACAT activity. Such agents can include enzymes that are involved in catabolism of ACAT, or enzymes that modify ACAT in a manner that reduces Aβ production. The agents may include enzymes that are involved in production of HtrA2 that modify HtrA2 in a manner that reduces Aβ production. The agents also can include molecules (such as RNAi or siRNA molecules) that reduce the level of enzymes involved in ACAT synthetic pathways. Thus, these methods include gene therapy applications.

In certain embodiments, the method for treating a subject with a disorder characterized by abnormal levels of Aβ production involves administering to the subject an effective amount of a nucleic acid molecule to treat the disorder. In certain of these embodiments, the method for treatment involves administering to the subject an effective amount of an antisense, RNAI, or siRNA oligonucleotide to reduce the level of an ACAT protein and thereby, treat the disorder. An exemplary molecule for modulating the levels of ACAT is a siRNA molecule that is selective for the nucleic acid encoding an ACAT protein. Alternatively, the method for treating a subject with a disorder characterized by abnormal levels of Aβ production involves administering to the subject an effective amount of an ACAT protein (or the nucleic acid that encodes such a protein) that has a reduces ACAT activity level, in order to treat the disorder.

In yet another embodiment, the treatment method involves administering to the subject an effective amount of a binding polypeptide (e.g., antibody, or antigen-binding fragment thereof) to modulate binding between one or more proteins of the invention and, thereby, treat the disorder. In some embodiments, the treatment methods involve administering to the subject an effective amount of a binding polypeptide to increase the level of ζ cleavage of APP and reduce the production of Aβ. In some embodiments, the treatment methods involve administering to the subject an effective amount of a binding polypeptide to decrease ACAT activity and decrease the production of Aβ. In certain embodiments, the binding polypeptide is an antibody or an antigen-binding fragment thereof.

Various techniques may be employed for introducing ζ cleavage-modulating compounds of the invention to cells or tissues, depending on whether the compounds are introduced in vitro or in vivo in a host. In some embodiments, the ζ cleavage-modulating compounds target neuronal cells and/or tissues. Thus, the ζ cleavage-modulating compounds can be specifically targeted to neuronal tissue (e.g. neuronal cells) using various delivery methods, including, but not limited to: administration to neuronal tissue, the addition of targeting molecules to direct the compounds of the invention to neuronal cells and/or tissues. Additional methods to specifically target molecules and compositions of the invention to brain tissue and/or neuronal tissues are known to those of ordinary skill in the art.

In some embodiments of the invention, a cleavage-modulating compound of the invention may be delivered in the form of a delivery complex. The delivery complex may deliver the ζ cleavage-modulating compound into any cell type, or may be associated with a molecule for targeting a specific cell type. Examples of delivery complexes include a ζ cleavage-modulating compound of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell specific receptor). Some delivery complexes may be sufficiently stable in vivo to prevent significant uncoupling prior to internalization by the target cell. However, the delivery complex can be cleavable under appropriate conditions within the cell so that the ζ cleavage-modulating compound is released in a functional form.

An example of a targeting method, although not intended to be limiting, is the use of liposomes to deliver a ζ cleavage-modulating compound of the invention into a cell. Liposomes may be targeted to a particular tissue, such as neuronal cells, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

Liposomes are commercially available from Life Technologies, Inc., for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications.

When administered, the ζ cleavage-modulating compounds (also referred to herein as therapeutic compounds and/or pharmaceutical compounds) of the present invention are administered in pharmaceutically acceptable preparations. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. Preferred components of the composition are described above in conjunction with the description of the pharmacological agents and/or compositions of the invention.

A pharmacological agent or composition may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The therapeutics of the invention can be administered by any conventional route including injection or by gradual infusion over time. Various modes of administration will be known to one of ordinary skill in the art which effectively deliver the pharmacological agents of the invention to a desired tissue, cell, or bodily fluid. The administration methods include: topical, intravenous, oral, inhalation, intracavity, intrathecal, intrasynovial, buccal, intraperitoneal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration (e.g., intra-organ) and the like vary from those presented herein.

The therapeutic compositions may conveniently be presented in unit dosage form and may be prepared by any of the methods well known in the art of pharmacy. All methods include the step of bringing the compounds into association with a carrier that constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US95/03307 (Publication No. WO 95/24929, entitled “Polymeric Gene Delivery System”). PCT/US95/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in PCT/US95/03307. The polymeric matrix may be in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers that can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993: 26: 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of established neurological disorder conditions as well as subjects at risk of developing a neurological disorder. “Long-term” release, as used herein, means that the implant is constructed and arranged to deliver therapeutic levels of the active ingredient for at least 7 days, and preferably at least 30-60 days or more. The implant may be positioned at or near the site of the neurological damage or the area of the brain or nervous system affected by or involved in the neurological disorder. Long-term sustained release implants are well known to those of ordinary skill in the art and include some of the release systems described above.

Some embodiments of the invention include methods for treating a subject to reduce the risk of a disorder associated with abnormal levels of Aβ production, HtrA2 activity, and/or ACAT activity. The methods involve selecting and administering to a subject who is known to have, is suspected of having, or is at risk of having an abnormal level of Aβ production, HtrA2 activity, and/or ACAT activity, a ζ cleavage-modulating compound, an HtrA2 activity-modulating compound, and/or an ACAT activity-modulating compound for treating the disorder. Preferably, the ζ cleavage-modulating compound, HtrA2 activity-modulating compound, and/or ACAT activity-modulating compound is a compound for enhancing ζ cleavage activity, a compound for enhancing HtrA2 activity, and/or a compound for inhibiting ACAT activity and is administered in an amount effective to increase ζ cleavage, increase HtrA2 activity, and/or reduce ACAT activity and therefore reduce production of Aβ.

Another aspect of the invention involves reducing the risk of a disorder associated with abnormal levels of Aβ production, HtrA2 activity, and/or ACAT activity, by the use of treatments and/or medications to modulate levels of ζ cleavage of APP, HtrA2 activity, and/or ACAT activity thereby reducing, for example, the subject's risk of an Aβ production-associated disorder.

In a subject determined to have an Aβ production-associated disorder, an effective amount of a ζ cleavage-modulating compound is that amount effective to increase levels of ζ cleavage, increase levels of HtrA2 activity, and/or decrease ACAT activity in a subject and therefore reduce Aβ accumulation in the subject. For example, in the case of Alzheimer's disease an effective amount may be an amount that inhibits (reduces) the levels of ACAT activity and/or an amount that enhances (increases) ζ cleavage of APP and/or an amount that increases HtrA2 activity.

A response to a prophylatic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has Alzheimer's disease, and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to modulate Aβ accumulation, and/or the levels of ζ cleavage of APP, HtrA2 activity, and/or ACAT activity and reduce, prevent, or eliminate Aβ production. For example, testing can be performed to determine the level of ζ cleavage of APP and/or the production of Aβ in subject's tissue and/or cells. Additional tests useful for monitoring the onset, progression, and/or remission, of Aβ production-associated disorders such as those described above herein, are well known to those of ordinary skill in the art. As would be understood by one of ordinary skill, for some disorders (e.g. Alzheimer's disease) an effective amount would be the amount of a pharmacological agent of the invention that decreases the levels of ACAT activity and/or increases the level of ζ cleavage of APP to a level and/or activity that diminishes the disorder, as determined by the aforementioned tests. In the case of HtrA2, it would be understood by one of ordinary skill that for some disorders (e.g. Alzheimer's disease) an effective amount would be the amount of a pharmacological agent of the invention that increases the level of HtrA2 activity and increases the level of ζ cleavage of APP to a level and/or activity that diminishes the disorder, as determined by the aforementioned tests.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

The pharmaceutical compositions used in the foregoing methods preferably are sterile and contain an effective amount of a pharmacological agent for producing the desired response in a unit of weight or volume suitable for administration to a patient.

The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent of the invention may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases including Aβ production-associated disorders of the invention. Thus, this invention is intended for use in husbandry and veterinary medicine as well as in human therapeutics.

The invention will be more fully understood by reference to the following examples. These examples, however, are merely intended to illustrate the embodiments of the invention and are not to be construed to limit the scope of the invention.

### EXAMPLES

### Example 1

Introduction

We used genetic disruption of ACAT-1 to identify the molecular events leading to decreased Aβ generation. We found that one or more membrane proteins bind to the N-terminal domain of APP in a cholesterol-sensitive way, inhibiting β- and α-cleavage of APP while directing APP toward a non-amyloidogenic endosomal proteolytic pathway involving a novel ζ-cleavage.

Methods

Cell Culture and Antibodies

Wild-type (WT) and cholesterol mutant (AC29 and 25RA) CHO cell lines were grown in Dulbecco's Modified Eagle Medium (DMEM) and Nutrient Mixture F-12 Ham (Sigma-Aldrich, St. Louis, Mo.), respectively. Media were supplemented with 10% (v/v) FBS (Atlanta Biologicals, Norcross, Ga.), 1% (v/v) L-Glutamine-Penicillin-Streptomycin solution (Sigma-Aldrich), and 0.4% (v/v) G418 Sulfate (Calbiochem, La Jolla, Calif.) as selection marker for cells stably transfected with cDNA constructs expressing APP751 or APP(ES/AA), and hygromycin for C470-V5/His in a pSec Tag/FRTN5/His TOPO expression vector (Invitrogen, Carlsbad, Calif.). Cells were cultured at 37° C. in a water-saturated air/CO₂ atmosphere.

The following antibodies against APP were used throughout this study: 22C11 (monoclonal, against N-terminus; Chemicon International, Temecula, Calif.); 369 (monoclonal, against C-terminus; generous gift from Dr. Sam Gandy, Farber Institute for Neuroscience, Thomas Jefferson University, Philadelphia, Pa.); C7 and C8 (against C-terminus; generous gift from Dr. Dennis J. Selkoe, Harvard Medical School, Boston, Mass.); 4G8 (monoclonal, against residues 18-25 of Aβ; Endogen, Woburn, Mass.); 6E10 (monoclonal, against residues 1-17 of Aβ; Senetek, St. Louis, Mo.). Antibodies against calreticulin (ER marker), GM130 (Golgi marker), and EEA-1 (early endosomes) were obtained from StressGen Biotechnologies Corp. (Victoria BC, Canada) and Transduction Laboratories (Lexington, Ky.). Anti-V5 antibody was from Invitrogen (Carlsbad, Calif.).

Additional materials used in this study include the γ-secretase inhibitor DAPT (generous gift from Dr. Rudolph E. Tanzi, Massachusetts General Hospital, Charlestown, Mass.), the β-secretase inhibitor Z-VLL-CHO (Calbiochem, La Jolla, Calif.), ALLN (Calbiochem), lactacystin (Sigma-Aldrich), ammonium chloride (Sigma-Aldrich), chloroquine (Sigma-Aldrich), methyl-(3-cyclodextrin (Sigma-Aldrich), and mevastatin (Sigma).

Cholesterol Determinations

For the determinations of intracellular pools of FC and CE, cells were first incubated ad equilibrium (for 3 days) in the presence of [¹⁴C]Acetic acid (57.0 mCi/mmol) (Amersham Life Science, Elk Grove, Ill.), then washed twice in PBS and extracted in chloroform/methanol (2:1; v/v). The chloroform phase was dried, resuspended again in chloroform, and applied with standards to a Silica Gel-G (EM Science, Gibbstown, N.J.) Thin Layer Chromatography (TLC). Plates were developed in hexane/ethyl ether/acetic acid (87:20:1, v/v) and visualized with 12 vapor. Spots were scraped and counted in a liquid scintillation counter.

In Vitro Reconstitution

Full-length APP was purified from either WT CHO or AC29 stably transfected cells using a Q Sepharose Fast Flow column (Pharmacia LKB, Uppsala, Sweden). Briefly, cell extracts were adjusted to 150 mM NaCl and loaded onto a Q-Sepharose column equilibrated in 23 mM Tris-pH 7.6, 4.7 mM EDTA, 150 mM NaCl, 0.24% Triton X-100. The column was washed with 350 mM NaCl in 23 mM Tris-pH 7.6, 4.7 mM EDTA, 0.24% Triton X-100 and then eluted with 4 column-volumes of 50 mM Tris-pH 8, 1 M NaCl. Purified APP was reconstituted into native intact membrane vesicles from either the ER or Golgi/endosomal compartments from WT CHO and AC29 untransfected cells. Latency of the vesicle preparations was analyzed using either the mannose-6-phosphate (Guillen, E., et al., (1995) Biochemistry 34: 5472-5476) or the sialyltransferase assays (Puglielli, L., et al., (1999b) J Biol Chem 274: 4474-4479). In the case of fractions containing Triton X-100, the detergent was removed using Extracti-Gel D Detergent Removing Gel (Pierce, Rockford, Ill.) immediately before the in vitro reconstitution. Reconstitution was achieved with three cycles of freeze-thawing in either acetone-dry ice or liquid nitrogen bath (Puglielli, L., et al., (1999) J Biol Chem 274: 35596-35600; Puglielli, L., et al., (1999a) J Biol Chem 274: 12665-12669). Reconstituted vesicles were incubated at 35° C. for 45 min. For the proteolytic digestion, reconstituted vesicles were co-incubated with chymotrypsin (1 mg/mg Golgi protein) in the presence or absence of 0.02% Triton X-100. Digestion was halted by adding anti-chymotrypsin specific inhibitor (1 mg inhibitor for every mg of protease). Chymotrypsin and its specific inhibitor were from Sigma. For the in vitro reconstitution of APPC470-V5/His, the protein was purified using a nickel-nitrilotriacetic acid (Ni-NTA) Agarose column (Qiagen, Valencia, Calif.) prior to reconstitution into native vesicles from the endosomal compartment.

Subcellular Fractionation

Cells were scraped and homogenized through a ball-bearing homogenizer. Homogenates were centrifuged at 1,500 r.p.m. for 20 min at 4-8° C. Postnuclear supernatants (PNS) were collected, applied on a 8-34% Nycomed OptiPrep (Invitrogen) continuous gradient and centrifuged at 27,000 rpm in a SW41 rotor for 18 h at 4° C. 0.8 ml fractions were collected.

Biotinylation of Cell Surface Proteins

To label cell surface proteins, growing cells were incubated with EZ-Link™ Sulfo-NHS-LC-Biotin (Pierce, Rockford, Ill.) for 45 min at 15° C. Cells were then washed twice in PBS, scraped, and lysed. Biotinylated proteins were separated from nonbiotinylated proteins using BioMag Streptavidin beads (Qiagen, Valencia, Calif.), electrophoresed on 4-12% Bis-Tris gel (Invitrogen, Carlsbad, Calif.), and probed with C7 antibody.

Aβ Determinations

For Aβ determination, APPwt and APPC470 stably transfected cells were grown in 6-well plates (Becton Dickinson Labware, Franklin Lakes, N.J.). Once ˜80-90% confluent, cells were washed in PBS and incubated in 1 ml of fresh medium for 24 hours. Secreted Aβₜₒₜₐₗ and Aβ₄₂ were quantitated by standard sandwich ELISA (Aβ ELISA Core Facility, Center for Neurological Diseases, Harvard Institutes of Medicine, Harvard Medical School).

Results

ACAT Inhibition Correlates with Two Novel N-terminal Cleavages of APP

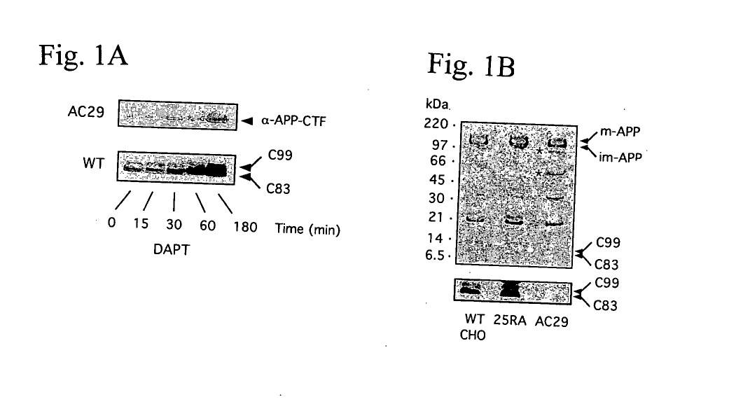

Inhibition of ACAT activity reduces the generation of Aβ and the APP-CTFs C99 and C83 (Puglielli, L., et al., (2001) Nat Cell Biol 3: 905-912). Specifically, the decrease in Aβ generation is correlated with reduced C99, the β-cleavage product of APP, since C83 does not contain full-length Aβ. To assess the extent to which β- and α-cleavages were reduced in AC29 cells stably transfected with APP, we inhibited further processing of these fragments by γ-secretase. The γ-secretase inhibitor DAPT increased C83 but not C99 levels, indicating that β cleavage of APP is undetectable in AC29 cells (FIG. 1A). Parallel reductions in both C83 and C99 levels suggest that an event upstream of cleavage likely controls the amyloidogenic processing of APP in an ACAT-regulated manner.

ACAT inhibition in AC29 cells could direct APP toward new pathways of proteolytic processing, perhaps resulting in reduced C83 and C99 generation. Therefore, we compared APP-CTF fragments produced in wild-type (WT) CHO, 25RA (AC29 parental cell line), and AC29 cells stably transfected with APP. Using C7, an antibody directed against amino acids 676-695 of the splice-form of APP containing 751 amino acids, (APP751) (Podlisny, M. B., et al., (1991) Am J Pathol 138: 1423-1435; Puglielli, L., et al., (2001) Nat Cell Biol 3: 905-912), we detected two major and novel APP-CTFs in AC29 cells, when compared to either WT CHO or 25RA cells (FIG. 1B, asterisks). These two new fragments, appearing as bands with molecular masses of ˜55- and ˜85-kDa, were also visible with 6E10 (against amino acids 1-17 of the A region), 4G8 (against amino acids 18-25 of the Aβ region), and 369 (against the C-terminus of APP) antibodies, but not with 22C11 antibody (against the N-terminus of APP). Interestingly, C8, an antibody directed against the same epitope as C7, did not detect these bands, perhaps due to conformational specificities. As we previously reported (Puglielli, L., et al., (2001) Nat Cell Biol 3: 905-912), both C99 and C83 were almost completely absent in AC29 cells (FIG. 1B).

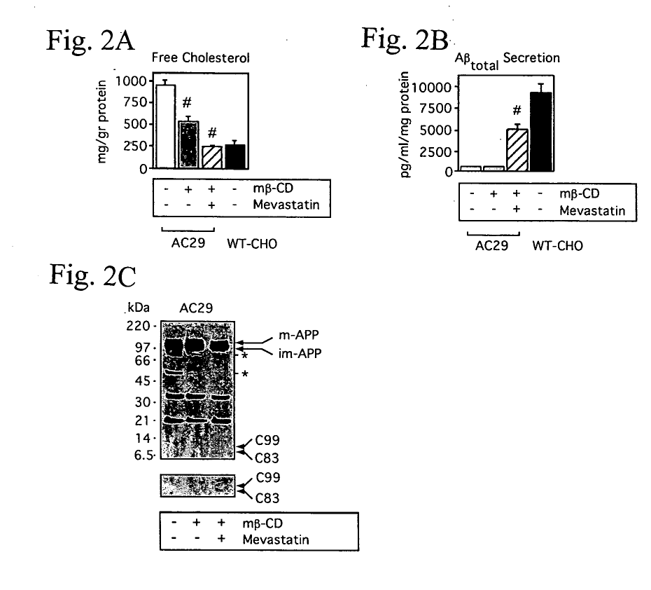

To establish whether the generation of the 55- and 85-kDa APP-CTFs was affected by FC in the absence of changes in CEs, we depleted AC29 cells of cholesterol using a combination of methyl β-cyclodextrin (m#-CD), a sterol-binding molecule, and mevastatin, an HMG-CoA reductase inhibitor. In our studies, mβ-CD was used for 24 hours to allow for cellular cholesterol to reach equilibrium. Cell viability was not affected, as assessed by the release of the cytosolic enzyme lactate-dehydrogenase (LDH) into the media. Mevastatin alone did not induce significant changes in cholesterol levels, whereas mβ-CD alone reduced, but did not normalize FC levels (FIG. 2A). Only the combination of mβ-CD plus mevastatin was able to normalize FC (FIG. 2A; compare to WT CHO cells). Normalization of FC levels was followed by a marked increase in both the secretion of Aβ into the media (FIG. 2B) and the steady-state levels of C99 and C83 in cells (FIG. 2C), indicating that FC levels are responsible, at least in part, for the reduced generation of Aβ observed in AC29 cells. However, it is important to note that normalized FC in AC29 cells only recovered 50% of the Aβ secreted by WT CHO cells (FIG. 2B), the other 50% still missing presumably due to the absence of CE synthesis in these cells. Both ˜55- and ˜85-kDa APP-CTFs were undetectable after successful normalization of FC (FIG. 2C).

Thus, in two different cellular models (untreated AC29 cells and mM-CD plus mevastatin-treated AC29 cells) we consistently observed a tight correlation between the appearance of the ˜55- and ˜85-kDa APP-CTFs, and reduced levels of C99, C83, and Aβ.

The N-terminus of APP Mediates the ACAT-Sensitive Reduction of Aβ Generation

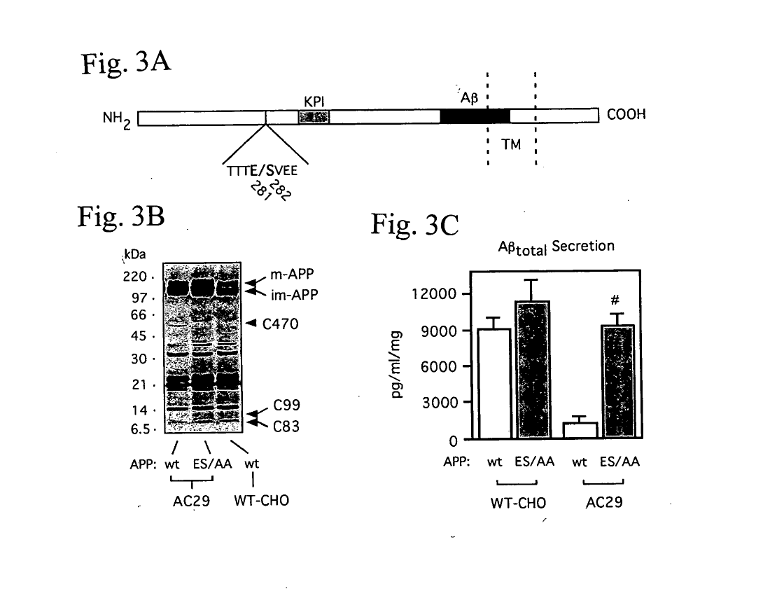

Since the ˜55-kDa fragment consistently appeared as the most prominent band of the two novel APP-CTFs, we further characterized the role of this fragment in the ACAT-mediated regulation of Aβ generation. For convenience, we named this novel cleavage of APP “ζ-cleavage”, to be serially consistent with other well-known proteolytic cleavages of APP. N-terminal sequencing and amino acid composition analysis revealed that the ˜55-kDa CTF begins at Ser282 of APP, yielding a CTF harboring 470 amino acids from the 751 amino acid splice form of APP (FIG. 3A). We named this new APPCTF “C470” in analogy to other well-characterized APP-CTFs, e.g. C83 and C99. “C470” is also referred to herein as “APP_{C470}”. Attempts to sequence the ˜85-kDa APP-CTF were unsuccessful because of very low recovery of this protein, most likely due to its molecular instability.

To establish whether ζ-cleavage of APP could modulate Aβ generation, we stably transfected AC29 cells with a cDNA construct harboring two point-mutations in the ζ-cleavage site (FIG. 3A). The point-mutations E281A and S282A (ES/AA) abolished the generation of C470 while reactivating both β- and α-cleavages of APP in AC29 cells (FIG. 3B). More importantly, the cells recovered their ability to produce and secrete Aβ (FIG. 3C). Interestingly, stable transfection of WT CHO cells with APP (ES/AA) also produced a modest increase in Aβ secretion (FIG. 3C). These data suggest that the ζ protease is constitutively active in WT CHO cells, despite undetectable C470 in these cells. The above results indicate that APP containing the point-mutations E281A and S282A is insensitive to genetic disruption of ACAT that would normally reduce Aβ generation.

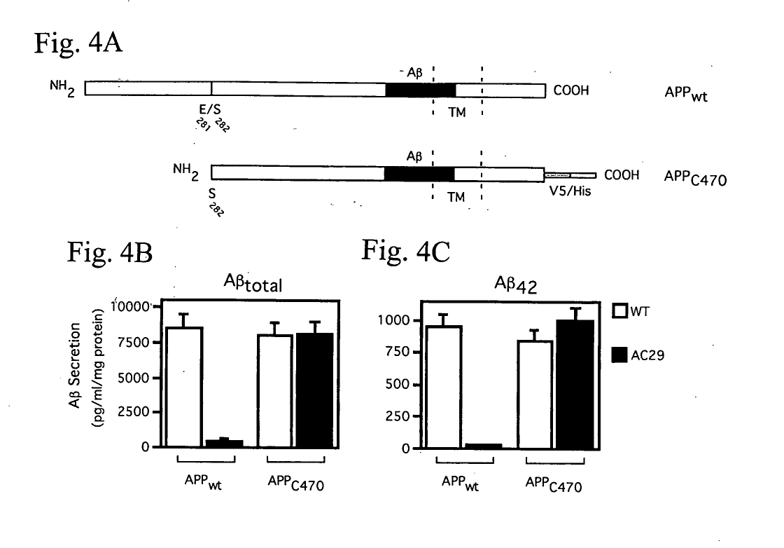

To further characterize the functional significance of the ζ-cleavage, we stably transfected both WT CHO and AC29 cells with a deletion mutant form of APP, missing amino acids 1-281. The resulting cDNA construct expressed C470 with a V5-His tag at its C-terminal tail (FIG. 4A). As expected, processing of APPC470 was normal in WT CHO cells in terms of Aβ, C99, and C83 generation (FIG. 4B, C). Surprisingly, however, AC29 cells stably transfected with APPC470 recovered the ability to generate Aβ and cleave APP at both α- and β-sites (FIG. 4B, C), indicating that C470 can act as a substrate for α- and β-secretases, independent of ACAT activity.

Taken together with the previous data, these results suggest that ACAT-sensitive processing of APP and Aβ generation requires both the N-terminal 1-281 amino acids and cleavage of APP in cells.

ζ-Cleavage of APP Occurs in the Endosomal Compartment

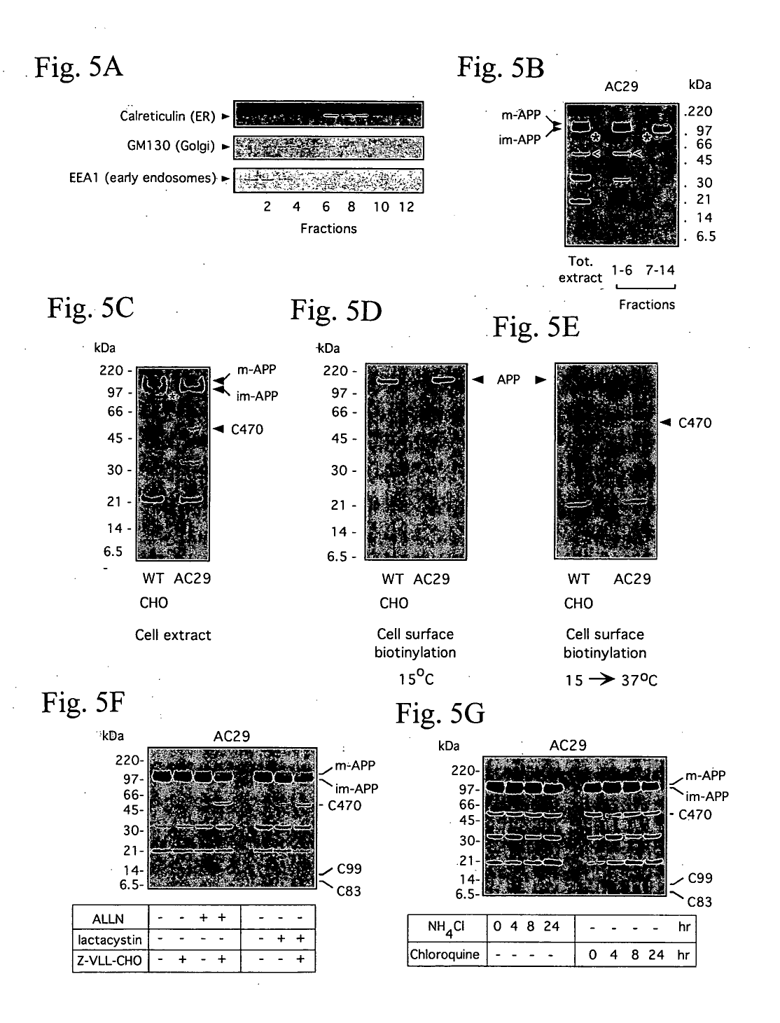

Since C470 contains the Aβ sequence and only lacks the first 281 amino acids of APP, ζ-cleavage likely takes place either in the lumen of intracellular organelles or at the extracellular surface. AC29 cells have no obvious defect in APP expression, maturation, or transport along the secretory/endocytic compartments (Puglielli, L., et al., (2001) Nat Cell Biol 3: 905-912). However, it is possible that a small percentage of APP is mislocalized in AC29 cells, due perhaps to altered protein interactions at the N-terminal domain of the protein. In order to determine the cellular compartment for ζ-cleavage, intracellular membranes obtained from AC29 cells stably transfected with APP were separated on an OptiPrep continuous gradient. C470 was only detected in the fractions corresponding to the endosomal compartment and Golgi apparatus, whereas the ˜85-kDa APP-CTF was detected in those corresponding to the ER (FIG. 5A, B). A Nicodenz continuous gradient, which separates membranes from the Golgi and the endosomal compartments more efficiently, revealed that C470 migrates with markers of both the endosomal and late (trans) Golgi compartments, but mainly associates with endosomes. To confirm that ζ-cleavage requires cell surface exposure and re-internalization into endosomes, we performed cell surface biotinylation experiments. Biotinylation of cell surface proteins at 15° C. did not reveal biotinylated C470 (FIG. 5C) suggesting that ζ-cleavage of APP only occurs after re-internalization of cell surface APP. Both exo- and endo-cytosis are blocked at 15° C. but are immediately reactivated once cells are switched back to permissive temperatures. Indeed, biotinylated C470 could be detected once cells were replaced at 37° C. after biotinylation (FIG. 5C), further supporting that ζ-cleavage of APP occurs following re-internalization of cell surface APP in AC29 cells. These data also show that the small percentage of APP that undergoes ζ-cleavage and fails to get processed by α- and β-secretases is exposed to the media on the cell surface and correctly undergoes endocytosis. Small changes in endosomal compartmentalization, causing BACE1 (β-secretase) to lose access to APP, could possibly account for our results. Alternatively, BACE1 and α-secretase may be inactive in cells with reduced ACAT activity.

To assess whether C470 undergoes proteasomal or lysosomal degradation, AC29 cells stably transfected with APP were first treated with the proteasome inhibitor ALLN, in the presence or absence of β-secretase inhibitors. ALLN alone increased the steady state levels of C99 but not C83 (FIG. 5D). This effect was completely reversed when ZVLL-CHO, a selective inhibitor of BACE1 activity (Abbenante, G., et al., (2000) Biochem Biophys Res Commun 268: 133-135), was used in addition to ALLN. The combined use of ALLN and Z-VLL-CHO produced a marked increase in the steady-state levels of C470, which was not evident when Z-VLL-CHO was used alone (FIG. 5D). These data suggest that when proteosomal degradation of C470 is inhibited by ALLN, C470 can still be processed by BACE1. Very similar results were obtained when lactacystin, a more selective inhibitor of proteasomes, was used instead of ALLN. In contrast, no effect was observed when the lysosomal inhibitors, chloroquine and ammonium chloride, were used (FIG. 5E).

Taken together, these results indicate that C470 is produced after internalization of cell-surface APP, most likely in the endosomal compartment, and is then rapidly degraded by the proteasome machinery. Whether C470 is targeted to the proteasomes from the endosomal compartment or after retro-transport to the ER remains to be determined.

Membrane Protein(s) Binding to the N-Terminus of APP Promote ζ-, While Reducing α- and β-Cleavages In Vitro

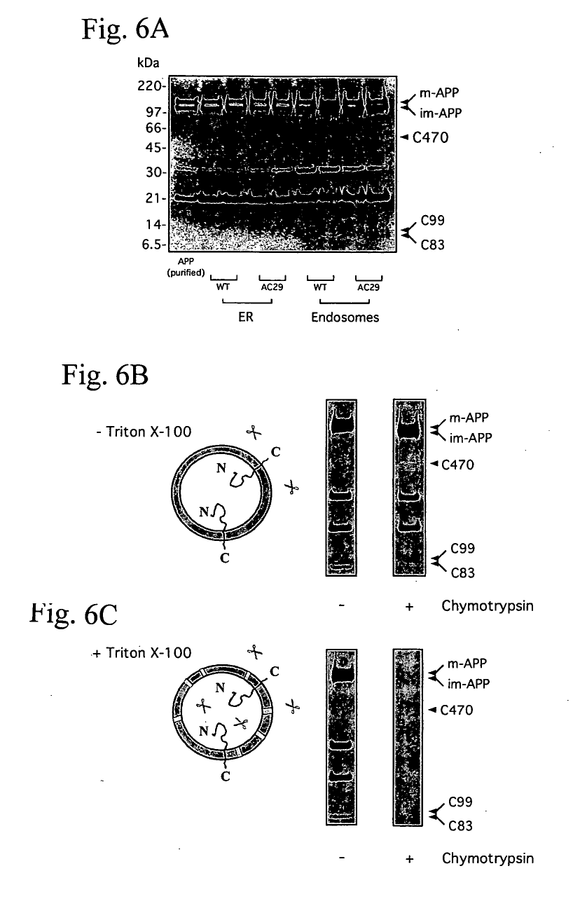

We next performed an in vitro reconstitution assay to assess whether α-, β-, and ζ-proteases were constitutively active in WT CHO and AC29 Golgi/endosomal compartments. APP was first purified from WT CHO cells using a Mono-Q Sepharose column, and then reconstituted into native ER and Golgi/endosomal membranes from untransfected WT CH0 or AC29 cells. C470, C99, and C83 were undetectable in the purified fraction of APP (FIG. 6A) and in the membrane preparations. Reconstitution was achieved after 3 cycles of freeze/thawing, as described for other membrane proteins (Puglielli, L., et al., (1999) J Biol Chem 274: 35596-35600; Puglielli, L., et al., (1999a) J Biol Chem 274: 12665-12669). Incubation for 45 minutes at 35° C. after reconstitution yielded newly produced APP-CTFs (FIG. 6A), which were not observed when purified APP was reconstituted after high temperature (100° C.) treatment or trypsin digestion. As expected, endosomal vesicles from WT CHO cells generated C99 and C83, while the same vesicles from AC29 cells produced C470 (FIG. 6A). Unexpectedly, however, WT CHO cells in vitro contained as much ζ-protease activity as AC29 cells, while AC29 cells harbored fully active α- and β-secretases (FIG. 6A). Control experiments show that reconstituted APP is inserted into the vesicles in the correct orientation (FIG. 6B). Briefly, we treated APP-reconstituted vesicles from WT CHO cells with chymotrypsin, in the presence or absence of the detergent Triton X-100. In the absence of the detergent, chymotrypsin is not able to cross the membrane and, therefore, cannot access the N-terminus of APP. This condition did not preclude the generation of C470 (FIG. 6B). In contrast, Triton X-100 permeabilization of the vesicles, which allows access of chymotrypsin to the lumen, led to the complete digestion of full-length APP and C470 (FIG. 6B). Triton X-100 pretreatment also abolished both C99 and C83 generation. These experiments show that the majority of reconstituted APP is inserted in the correct orientation with the N-terminal domain inside the lumen of the vesicles and that C470 is generated within the lumen of the vesicles.

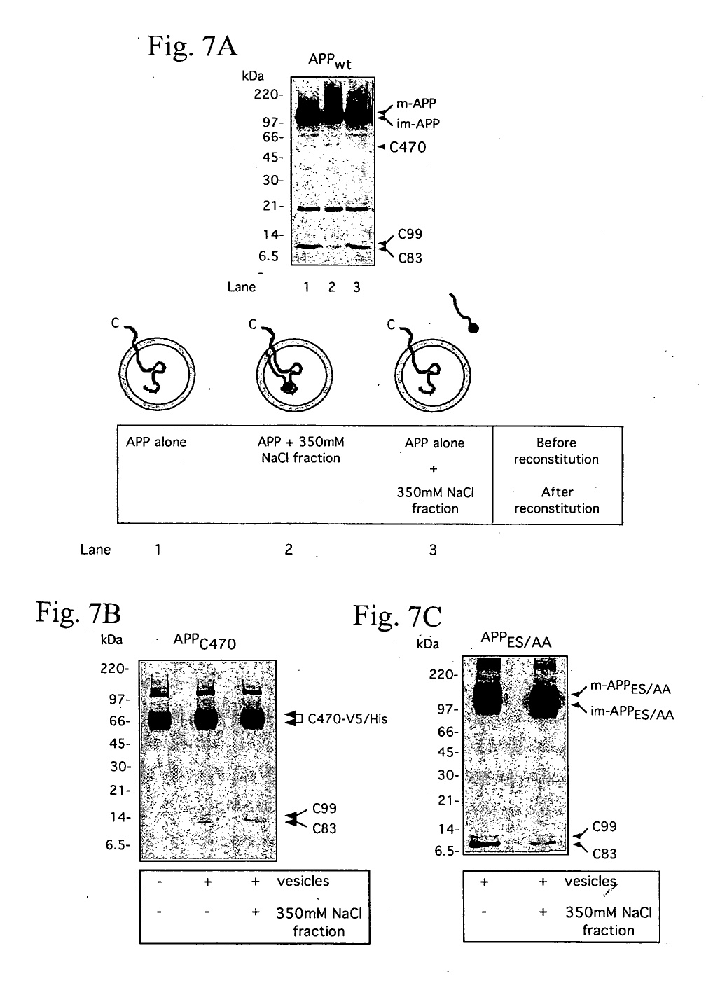

The surprising result that α-, β-, and ζ-proteases are all constitutively active in endosomal compartments of both WT and AC29 CHO cells suggests that access of these three proteases to APP is regulated differently in these two cell lines. One possibility is that a “factor” (lipid or protein) binding to the first 281 amino acids of APP determines whether APP is processed via the amyloidogenic or the sterol-regulated pathways. It is very likely that the association between APP and the putative regulatory “factor” was disrupted when we extracted and purified APP. The “factor” could conceivably either alter the trafficking of APP or directly modulate access of the proteases to their respective cleavage sites. To distinguish between these two mechanisms and to confirm the existence of this “factor”, we repeated the in vitro reconstitution assay in the presence of different elution fractions obtained during the biochemical purification of APP. Upon reconstitution of APP in endosomal membranes from WT CHO cells and incubation for 45 minutes at 35° C., we found that the 350 mM NaCl elution fraction from the Mono-Q Sepharose column was able to reduce both (α-, and β-cleavages of APP and increase the generation of C470 (FIG. 7A, lane 2). This effect was not observed when the same fraction was added after reconstitution (FIG. 7A, lane 3). In addition, high-temperature (100° C.) and trypsin digestion of the 350 mM NaCl fraction prior to in vitro reconstitution abolished its ability to inhibit α- and β-cleavages of APP, indicating that the “factor” is not a lipid, but rather a membrane protein(s) that requires reconstitution into the bilayer. Thus, our data suggest that one or more membrane proteins interact with APP and induce ζ-cleavage, while inhibiting α- and β-cleavages of the protein.

To assess the impact of the N-terminal 281 amino acids of APP and of ζ-protease cleavage on C99 and C83 levels in vitro, we repeated the previous in vitro reconstitution experiments using C470-V5/His, the ζ-protease cleavage product (see FIG. 4A), and APP (ES/AA), which cannot be cleaved at the E281/S282 site (see FIG. 3A). After reconstitution of C470-V5/His, C99 and C83 were not reduced by the 350 mM NaCl fraction indicating that the first 281 amino acids of APP were required to mediate the inhibitory effect of the membrane protein(s) in the 350 mM NaCl fraction on α- and O-cleavages (FIG. 7B). These in vitro results confirm our experiments in cells, showing that ACAT-mediated regulation of APP processing requires the N-terminus of APP. Reconstitution of APP (ES/AA) into intact native endosomal vesicles from WT CHO cells permitted both α- and β-cleavages of APP without the production of C470 (FIG. 7C). Both C99 and C83, however, were greatly reduced upon reconstitution of the 350 mM NaCl fraction (FIG. 7C), indicating that ζ-cleavage was not required for the inhibitory effect of the 350 mM NaCl fraction. These in vitro data with APP (ES/AA) contrast with our cell-based studies where ζ-cleavage was required for ACAT-mediated reduction of C99 and C83. One possible explanation for this contrast is that our ES/AA point-mutations introduced into the ζ-secretase cleavage site may have altered the conformation of APP in cells, which in turn could have affected its complex molecular interactions in the ER and/or its trafficking, resulting in failed binding to the regulatory membrane protein(s) and insensitivity to ACAT inhibition. However, the molecular conditions in the endosomal vesicles used in the in vitro assay were different from those in the ER of intact cells, resulting in successful interaction of APP(ES/AA) with the membrane protein(s) of the 350 mM NaCl fraction and decreased APP-CTF generation.

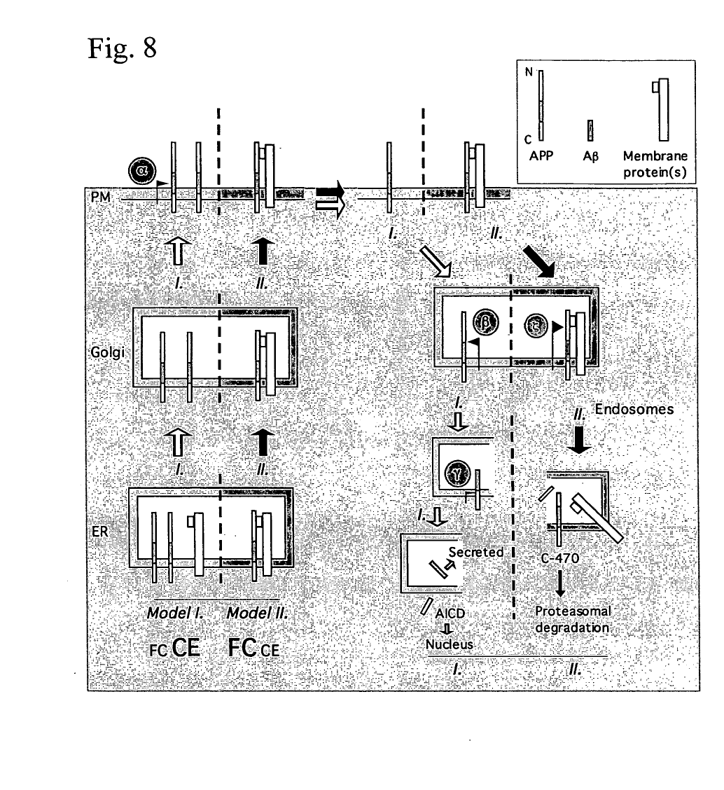

In conclusion, both cell-based and in vitro data support a model where ACAT inhibition and reduced CE favor the binding of one or more membrane proteins to the N-terminal 1-281 amino acids of APP. This interaction then results in reduced OL- and #-cleavages and increased ζ-cleavage of the protein, thus explaining the dramatic decrease of Aβ generation caused by inhibition of ACAT activity (FIG. 8).

FIG. 8 Model I (low FC/CE ratios and normal processing of APP by α- and β-secretases): illustrates that APP is a type I membrane protein with the N-terminus facing the extracellular milieu or the lumen of the organelles. Newly synthesized APP is translocated to the plasma membrane (PM) along the secretory pathway. The majority of cell surface APP is not immediately processed but re-internalized into the endosomal compartment and, eventually, sorted back to the PM as a typical cell-surface receptor. During this process, a fraction of APP can be cleaved by α- or β-secretase. The former is preferentially active at or close to the PM, whereas the latter is predominantly active in the late-Golgi apparatus and in the endosomal compartment. The CTFs generated by both these cleavages are further processed by γ-secretase and give rise to the cytoplasmic APP intracellular domain, or AICD, which enters the nucleus to take part in transcriptional regulation of specific genes. Only the β-CTF, or C99, gives rise to secreted Aβ.

FIG. 8 Model II (elevated FC/CE ratios and ζ-secretase processing of APP): illustrates that cholesteryl-esters (CEs) are generated in the ER from free cholesterol (FC) and fatty acids. ACAT1, an ER resident enzyme, catalyzes this reaction. After synthesis, CEs accumulate in the cytosol in the form of gel-like structures called “cytosolic droplets”. Our results indicate that a delicate balance between FC and CEs regulates the molecular interaction between APP and novel membrane protein(s). The most likely site of contact is the ER, given that this is the only membrane that contains both forms of cholesterol: FC and CE. ACAT1 inhibition causes net levels of CEs to decrease and FC to increase. This metabolic event allows for the molecular interaction of the N-terminus (amino acids 1-281) of APP with one or more membrane proteins. Our results suggest that this interaction does not influence APP trafficking in a detectable manner. Instead, it precludes both α- and β cleavages of APP, while allowing for ζ-cleavage in an endosomal compartment. The ζ protease is a novel protease that cleaves APP at Glu281, generating a 470 amino acid-long CTF (C470). Although C470 first appears in an endosomal compartment, it is not recycled to the PM, either because it lacks the N-terminus or because it rapidly undergoes proteasomal degradation. Thus, APP cleaved at the ζ site does not appear to undergo α-, β-, or γ-secretase-mediated processing, thereby not releasing Aβ into the media or the transcriptional regulator AICD into the cytoplasm.

Discussion

We have shown that ACAT activity regulates the biogenesis of Aβ by a mechanism requiring the N-terminal 1-281 amino acids of APP and involving a novel cleavage event that replaces f-secretase cleavage of APP. ACAT inhibition results in two new proteolytic cleavages in the N-terminal domain of APP, the most predominant of which occurs after Glu281 (here called ζ-cleavage). This activity was reconstituted in an in vitro system and shown to occur in the lumen of the endosomal compartment, independently of intracellular cholesterol distribution. We found that ACAT regulates the molecular interaction between APP and as of yet unidentified membrane protein(s). This interaction requires the first 281 amino acids of N-terminus of APP, and directly precludes both α- and β-site cleavages, resulting instead in ζ-cleavage of APP. Collectively, our data support the model illustrated in FIG. 8 and discussed below.

The activation of the sterol-regulated proteolysis of APP requires exchange of cholesterol from the CE to the FC pool. Indeed, both biochemical and metabolic experiments in the cholesterol mutant cell lines indicate that increased levels of FC activate the generation of C470 only when CE levels are reduced. This is particularly evident in 25RA cells under ACAT inhibition (FIG. 1), where C470 could not be detected after one week of treatment, even when FC levels were as much as two-fold higher than in untreated cells. Similar levels of FC were sufficient to generate C470 in the absence of CEs (AC29 in FIG. 2; see mβ-CD alone). C470 was visible in 25RA cells after 3 weeks of ACAT inhibition, which produced a net decrease in CE levels associated with increased FC. The reason for this “metabolic requirement” is consistent with the existence of novel protein(s) that regulate APP processing in a “cholesterol-sensitive” manner. Moreover, different affinities for FC and CE may ultimately control the interaction between APP and these novel protein(s). This binding would be stabilized by conditions that favor the accumulation of FC versus the generation of CE, producing a net increase in the FC/CE ratio. Under these conditions, the interacting protein(s) would inhibit the amyloidogenic proteolysis of APP while activating ζ-cleavage. In contrast, CE would reactivate the normal amyloidogenic pathway by displacing the binding between APP and the interacting protein(s). Changes in cholesterol distribution in membranes other than the ER might also affect intracellular membrane dynamics and APP processing without the activation of the ζ-pathway. It is interesting to notice in this regard that statin treatment of cells, resulting in reduction of both CEs and FC, inhibits γ-secretase activity (Fassbender, K., et al., (2001) Proc Natl Acad Sci USA 98: 5856-5861), while impaired NPC1 function, resulting in high FC/low CEs in the endosomal compartment, also inhibits γ-secretase function (Runz, H., et al., (2002) J Neurosci 22: 1679-1689; Burns, M., et al., (2003) J Neurosci 23: 5645-5649).

When compared to FC, CE levels in the majority of mammalian tissues are low. In extracts from whole brains, CEs are almost undetectable and generally regarded as being lower than 5% of total brain cholesterol, while CEs constitute 25% of total cholesterol in mouse cortical neuronal populations (manuscript in preparation). Both forms of cholesterol, CE and FC, are synthesized in the ER membrane but then rapidly removed after synthesis: CE is stored into cytosolic lipid droplets, while FC is translocated to the PM. It is worth stressing that in normal conditions, the PM contains ˜80 to 90%, while the ER membrane contains only ˜0.5-1% of total cell FC. Thus, the ER membrane harbors very low levels of both FC and CE and possesses sophisticated mechanisms to maintain it constantly low. The metabolic requirement for very low levels of FC and CE in the ER membrane likely allows for high selectivity and sensitivity, which is necessary for fine-tuning of SREBP and APP processing.

Our in vitro reconstitution experiments show that while both ζ-protease and the amyloidogenic proteolytic activities are active in the endosomal membrane fraction, they are mutually exclusive in cell cultures. One possible explanation for this is the existence of different membrane compartments where β-, and ζ-cleavage of APP occur. However, this is unlikely based on both genetic and biochemical studies in vitro showing a tight correlation between decreased β- and increased ζ-cleavage only in the presence of the N-terminal domain of APP. Our results show that the N-terminally truncated form of APP, C470, can still serve as an α- or β-secretase substrate. However, when C470 is generated in the endosomal compartment during ACAT inhibition, this does not occur. Once localized to a specific endosomal compartment, the N-terminus of APP may be required for recycling back to the cell surface for subsequent availability to α-secretase. Since it lacks the N-terminus, C470 cannot be sorted to the PM from the endosomal compartment and is instead degraded by the proteasome machinery. Lack of cell surface recycling and proteasomal degradation of C470 do not fully explain our results. We propose an additional role for regulatory membrane protein(s) that interact with the first 281 amino acids of APP. The molecular interaction between APP and the novel membrane protein(s) most likely occurs in the ER as the ER is the only intracellular membrane where both FC and CEs can be found. Some possibilities explaining how N-terminally binding protein(s) may tilt the balance from (β- (and α-) toward ζ-cleavage of APP include the following: APP may undergo a conformational change that precludes access of β-secretase to its target sequence; or, the N-terminally binding membrane protein(s) may directly mask the β-secretase target sequence; or, C470 may be rapidly degraded prior to β-cleavage. Finally and least likely, APP may be mistargeted away from the membrane compartment where (3-cleavage of APP occurs. Definitive answers to these possibilities will require purification and cloning of ζ-protease and the APP N-terminally binding protein(s).

Thus far, cholesterol has been shown to directly regulate the processing only of proteins implicated in the cholesterol homeostasis feedback system: HMG-CoA reductase, the key enzyme in cholesterol synthesis, and SREBPs, which regulate cholesterol synthesis and uptake. The resident ER protein Insig-1 directly regulates the proteasomal degradation of HMG-CoA reductase (Sever, N., et al., (2003) Mol Cell 11: 25-33), and indirectly modulates the proteolytic processing of SREBPs in the Golgi by strictly controlling the quantity of SCAP-SREBP complex that buds from the ER (Hua, X., et al., (1996) Cell 87: 415-426). Interestingly, the cytoplasmic domains of both SREBPs and APP are released and enter the nucleus to regulate transcription of specific genes (Brown, M. S., et al., (1997) Cell 89: 331-340; Brown, M. S., et al., (1999) Proc Natl Acad Sci USA 96: 11041-11048; Cao, X., et al., (2001) Science 293: 115-120; Kimberly, W. T., et al., (2001) J Biol Chem 276: 40288-40292; Scheinfeld, M. H., et al., (2003) Proc Natl Acad Sci USA 100: 1729-1734). In addition to both acting as transcription factors, SREBPs and APP are also both involved in two age-dependent disorders (atherosclerosis and AD), and both undergo proteolytic processing that has been highly conserved throughout evolution, from Drosophila melanogaster to humans (Nohturfft, A., et al., (2002) Science 296: 857-858; Puglielli, L., et al., (2001) Nat Cell Biol 3: 905-912: Tschape, J. A., et al., (2002) Embo J 21: 6367-6376). In the case of SREBPs, decreased ER cholesterol results first in a proteolytic event (Site 1 proteolysis) in the lumen of the Golgi that, in turn, activates a second cleavage (Site 2 proteolysis) that occurs in the Golgi membrane and releases the transcriptionally active N-terminal domains into the cytoplasm (reviewed in Brown, M. S., et al., (1997) Cell 89: 331-340). For APP, a high FC/CE ratio in the ER membrane induced by ACAT inhibition, reduces α- and β-cleavages thus decreasing levels of C83 and C99, the two major γ-secretase substrates. Cleavage at the γ-secretase site is regulated by the availability of γ-secretase substrates (Vassar, R., et al., (1999) Science 286: 735-741). Interestingly, in addition to Aβ, γ-secretase cleavage releases the transcriptionally active C-terminal domain of APP, called AICD (APP intracellular domain) (Cao, X., et al., (2001) Science 293: 115-120; Kimberly, W. T., et al., (2001) J Biol Chem 276: 40288-40292; Scheinfeld, M. H., et al., (2003) Proc Natl Acad Sci USA 100: 1729-1734). Thus, ACAT inhibition likely blocks the release of AICD. AICD production would then be inhibited at three levels. First, generation of APP C99 and C83, physiological secretase substrates, is strongly reduced. Second, C99 and C83 are replaced with C470, harboring a lumenal domain too large for efficient γ-secretase processing in the membrane (Struhl, G., et al., (2000) Mol Cell 6: 625-636). Third, C470 undergoes rapid degradation, perhaps further limiting its access to γ-secretase. Therefore, cholesterol feedback blocks ER exit of SREBPs, preventing cleavage and transcription, while promotes HMG-CoA reductase degradation; similarly, high FC/CE ratios block production of γ secretase substrates, preventing transcriptional regulation by AICD. It remains to be determined that the generation of C470 indeed correlates with decreased release of AICD, and, perhaps more interestingly, that the cytoplasmic domain of APP plays a major role in regulating aspects of cholesterol homeostasis.

Very recently genetic screening has identified a polymorphism in SOAT1, the gene that encodes ACAT-1, which is associated with low brain amyloid load and reduced risk for AD in the general population (Wolhmer, et al., (2003) Mol Psychiatry 8: 635-638). In addition, analysis of lipid metabolism in the spinal cord of patients affected by Amyotrophic Lateral Sclerosis (ALS) has revealed abnormal changes in sphingolipid metabolism together with abnormal accumulation of CE (Cutler, R. G., et al., (2002) Ann Neurol 52: 448-457). Therefore, in addition to atherosclerosis, CE metabolism appears to be linked to two different neurodegenerative disorders affecting central (AD) or peripheral (ALS) neurons. It is worth noting that alterations in cholesterol metabolism have also been linked to two additional neurological disorders, affecting the development of the brain during late embriogenesis (Smith-Lemli-Opitz syndrome) and early childhood (Niemann-Pick type C1). Cholesterol metabolism thus plays a crucial role in brain function during both youth and aging. With regard to AD related neurodegeneration, our results have uncovered a novel molecular pathway connecting FC/CE ratios with Aβ generation. Characterization of this pathway may lead to the development of new therapeutic strategies for the treatment and/or prevention of AD, based on reducing Aβ generation.

### Example 2

Identification of the Cleavage Protein as HtrA2 Protease

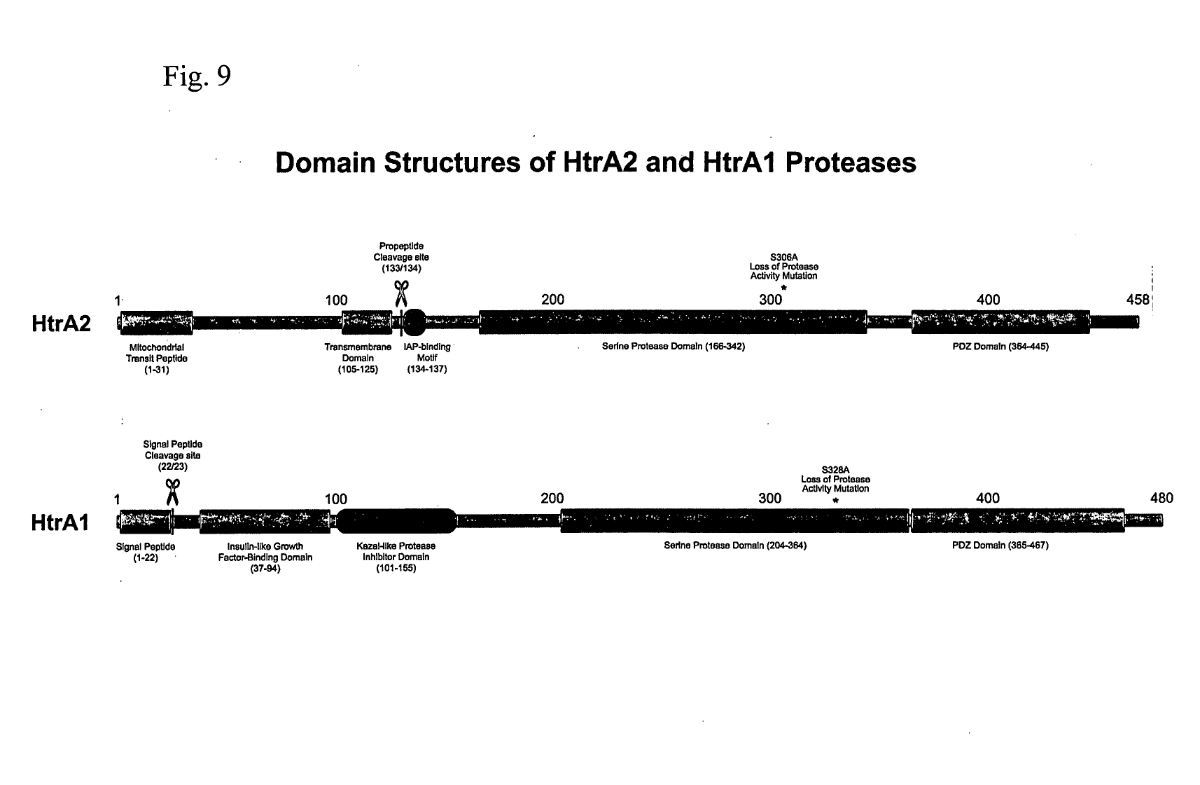

In a yeast two-hybrid assay, we found that the N-terminus of APP interacts with a protease called HtrA2. HtrA2/Omi belongs to a novel class of serine proteases containing a PDZ domain. FIG. 9 illustrates the domain structures of HtrA1 and HtrA2 proteases. We examined whether HtrA2 was a candidate for a sterol-regulated APP chaperone/protease.

Methods

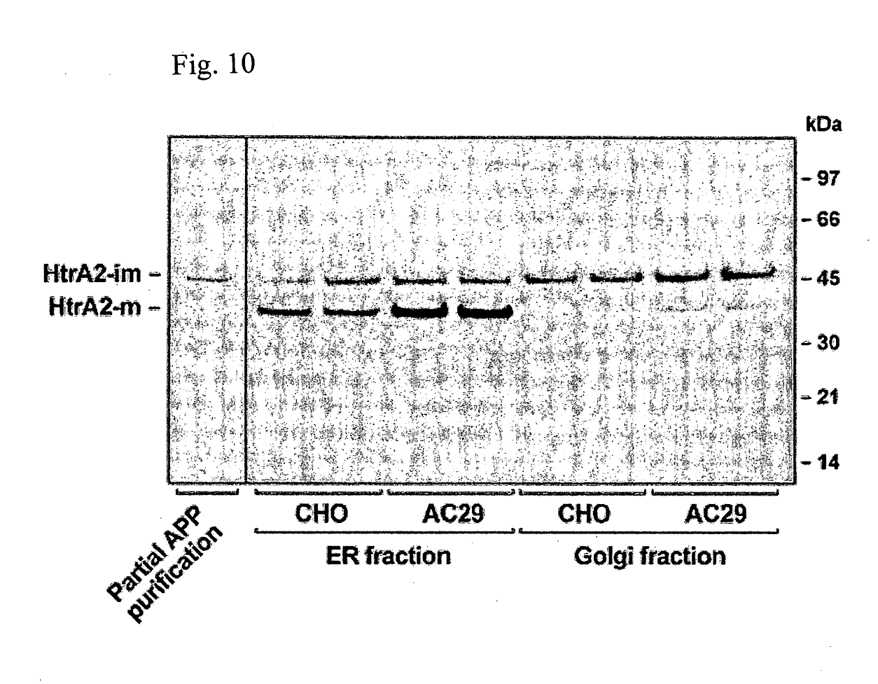

We determined that there were increased amounts of mature HtrA2 in the ER fraction of AC29 cells. Wild-type CHO cells and AC29 cells were grown to 70-80% confluency and scraped in extraction buffer containing 1% Triton X-100 and 0.25% NP-40. Postnuclear supernatants were fractionated in 8-34% Nycomed OptiPrep (GibcoBRL, Gaithersburg, Md.) continuous gradient by ultracentrifugation at 100,000 g for 18 hours at 4° C. in an SW41 rotor. Fractions were resolved on a 4-12% Bis-Tris SDS-PAGE gel (NuPage/Invitrogen, Carlsbad, Calif.), blotted on a nitrocellulose filter and analyzed for ER and Golgi markers calnexin and GM130, respectively. The blot (FIG. 10) was re-stained with rabbit anti-HtrA2 antibodies (R&D Systems, Minneapolis, Minn.) and visualized with enhanced chemiluminescence. Increased amounts of mature HtrA2 were found in the ER and Golgi fractions of AC29 as compared to the corresponding fractions from wild-type CHO cells. On the first lane, an aliquot from a partially purified APP₇₅₁ sample was resolved on the same gel. Staining with HtrA2 antibodies show that HtrA2 co-purifies with APP₇₅₁ in ion exchange chromatography (Q Sepharose; Amersham Biosciences, Piscataway, N.J.).

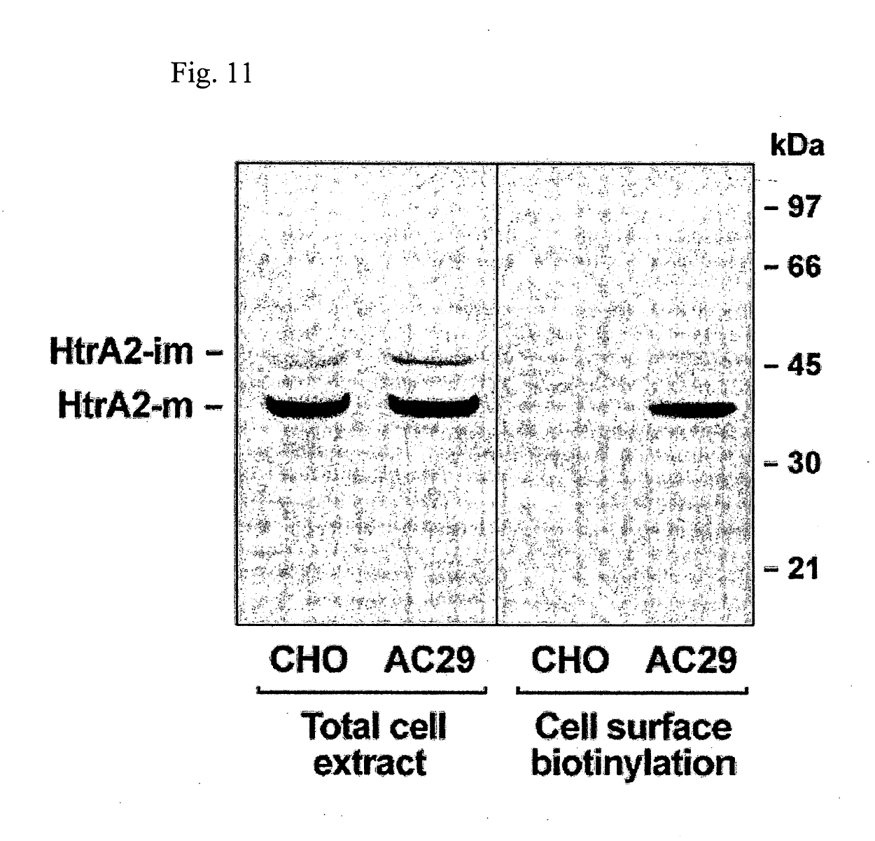

We have demonstrated HtrA2 localization in AC29 cells. FIG. 11 shows cell-surface localization of HtrA2 in AC29 cells. Wild-type CHO cells and AC29 cells were grown to 70-80% confluency and subjected to cell surface biotinylation. Cells were scraped in extraction buffer containing 1% Triton X-100 and 0.25% NP-40. Biotinylated proteins were captured from equal amounts of cell extract (normalized to protein concentration) with streptavidin magnetic beads and resolved on a 4-12% Bis-Tris SDS-PAGE gel (NuPage/Invitrogen) together with total cell extracts. Proteins were blotted on a nitrocellulose filter, stained with rabbit anti-HtrA2 antibodies (R&D Systems) and visualized with enhanced chemiluminescence. Both mature and immature forms of HtrA2 were biotinylated only in AC29 cells suggesting that HtrA2 was localized to the cell surface in AC29 but not in wild-type CHO cells. Similar amounts of HtrA2 were found in total cell extracts suggesting that HtrA2 expression was not significantly elevated in AC29 cells as compared to the wild-type CHO cells.

Discussion

HtrA2/Omi was originally identified as a mammalian homologue of the Escherichia coli protein HtrA or DegP, an important chaperone and protease in the bacterial periplasmic stress response system (Clausen et al., 2002. Mol. Cell 10, 443-455). In mammalian cells, HtrA2 has been shown to localize to the mitochondrial intermembrane space similar to the bacterial periplasmic space and has adopted a function as a regulator of apoptosis (Vaux & Silke, 2003 Biochem. Biophys. Res. Comm. 304, 499-504). When cells are exposed to apoptotic stimuli, the endoproteolytically processed mature form of HtrA2 translocates from mitochondria to the cytosol where it can promote apoptosis by antagonizing a class of proteins called inhibitors of apoptosis (IAP; Vaux & Silke, 2003). In addition to the mitochondria, HtrA2 has also been reported as localized to the ER and nucleus (Gray et al., 2000 Eur. J. Biochem. 267, 5699-5710).

Recent studies of HtrA2 knockout mice suggest that mammalian HtrA2 is likely to function as a protector against cell stresses in vivo in a similar manner to the bacterial DegS and DegP (Martins et al., 2004 Mol. Cell Biol. 24, 9848-9862). We determined that HtrA2 could serve as a chaperone binding to APP that under cellular stress could switch from chaperone to a protease that degrades misfolded APP. AC29 cells have elevated free (membrane) cholesterol level that may cause ER stress. In AC29 cells, levels of mature form of HtrA2 were elevated in the ER and Golgi as compared to the parental CHO cells (FIG. 10). Furthermore, HtrA2 localized to the cell surface in AC29 cells but not in the parental CHO cells (FIG. 11). Also we determined that HtrA2 co-purified with APP from mammalian cells (FIG. 10) and also co-immunoprecipitated with APP. In in vitro assays, recombinant HtrA2 degraded APP. Interestingly, HtrA2 bound to an APP region very close to the sterol-dependent cleavage site (E281/S282) (yeast two-hybrid assay) and also bound to the Aβ region of APP (Park et al., 2004 Neurosci. Lett. 357, 63-67).

Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method for identifying compounds that modulate amyloid precursor protein (APP) cleavage, comprising
providing a reaction mixture that comprises APP and/or a fragment thereof that includes a ζ-cleavage site, and protein(s) having ζ-cleavage activity,
contacting the reaction mixture with a test compound,
determining a level of ζ cleavage of APP in the absence and in the presence of the test compound, and
comparing the level of ζ cleavage in the absence and in the presence of the test compound, wherein a test compound that increases or decreases the level of ζ cleavage from the level of ζ cleavage in the absence of the test compound is a compound that modulates APP cleavage.

2. The method of claim 1, wherein the compound increases the level of ζ cleavage.

3. The method of claim 2, wherein the increase in the level of ζ cleavage is indicative of a reduction in Aβ prodution.

4. The method of claim 1, wherein the APP or fragment thereof is a modified APP or fragment thereof.

5. The method of claim 4, wherein the modified APP or fragment thereof is a polypeptide that is ζ cleavable.

6. The method of claim 4, wherein the APP or fragment thereof is a modified APP or fragment thereof that comprises a ζ-cleavage site and is ζ cleavable.

7. The method of claim 1, wherein the protein having ζ-cleavage activity is HtrA2.

8. The method of claim 1, wherein the level of ζ cleavage is determined using an antibody or antigen-binding fragment thereof that specifically binds to amyloid precursor protein (APP) and/or a fragment of APP.

9. The method of claim 8, wherein the antibody is selected from the group consisting of: 22C11, 369, C7, C8, and 6E10.

10. The method of claim 8, wherein the fragment of amyloid precursor protein (APP) is a fragment that comprises amino acid 281 and 282 of APP.

11. The method of claim 1, wherein the fragment of amyloid precursor protein (APP) comprises the N terminus of APP or the C terminus of APP.

12. The method of claim 11, wherein the N-terminus fragment of APP includes amino acid 281 and the C-terminus fragment of APP includes amino acid 282.

13. The method of claim 1, where in the fragment of amyloid precursor protein (APP) is APPC₄₇₀ or APP_{N1-281}.

14. The method of claim 8, wherein the antibody or antigen-binding fragment thereof is used in an ELISA assay.

15. The method of claim 8, wherein the antibody or antigen-binding fragment thereof is used in a Western blot assay.

16. The method of claim 1, wherein the reaction mixture is a cell.

17. The method of claim 1, wherein the compound that modulates ζ-cleavage inhibits or enhances HtrA2 activity.

18. The method of claim 1, wherein the compound that modulates ζ-cleavage inhibits or enhances acyl-coenzyme A:cholesterol acyltransferase (ACAT) activity.

19-21. (canceled)

22. An isolated fragment of amyloid precursor protein (APP) that comprises amino acid 281 and 282 of APP.

23-26. (canceled)

27. A method for identifying polypeptides that are G cleavable, comprising
providing a reaction mixture that comprises a candidate polypeptide suspected of being ζ-cleavable,
contacting the candidate polypeptide with a protein(s) having ζ-cleavage activity, and
determining a level of ζ cleavage of the candidate polypeptide, wherein the presence of ζ cleavage indicates that the candidate polypeptide is ζ cleavable.

28-46. (canceled)
